(19) United States
(12) Patent Application Publication
(10) Pub. No.: US 2021/0052528 A1
(43) Pub. Date: Feb. 25, 2021

(54) THE USE OF SGC ACTIVATORS AND SGC STIMULATORS FOR THE TREATMENT OF COGNITIVE IMPAIRMENT

(71) Applicant: Bayer Aktiengesellschaft, Leverkusen (DE)

(72) Inventors: Peter Sandner, Wuppertal (DE); Jos Prickaerts, AJ Maastricht (NL)

(73) Assignee: Bayer Aktiengesellschaft, Leverkusen (DE)

(21) Appl. No.: 17/051,060

(22) PCT Filed: Apr. 12, 2019
(86) PCT No.: PCT/EP2019/059390

§ 371 Date: Oct. 27, 2020

(30) Foreign Application Priority Data
Apr. 30, 2018 (EP) 18170049.3

(51) Int. Cl. A61K 31/192; A61K 31/506; A61K 31/437; A61K 31/445; A61K 31/27; A61K 31/55; A61K 31/13; A61P 25/28; A61K 45/06

(52) U.S. Cl. CPC A61K 31/192; A61K 31/506; A61K 31/437; A61K 31/445; A61K 45/06; A61K 31/55; A61K 31/13; A61P 25/28; A61K 31/27

(57) ABSTRACT

The present invention relates to sGC activators and sGC stimulators for use in the treatment of cognitive impairment in a mammal in need of such treatment, in particular for use in the treatment of vascular dementia.

The present invention relates to sGC activators and sGC stimulators for use in the treatment of cognitive impairment in a mammal in need of such treatment, in particular for use in the treatment of vascular dementia.

### BACKGROUND OF THE INVENTION

The cyclic nucleotides, cyclic adenosine monophosphate (cAMP) and cyclic guanosine monophosphate (cGMP) were discovered decades ago and represent one of the most important second messenger pathway within cells. It is well established that the regulation of intra-cellular cGMP pools has substantial impact on physiology and pathophysiology and is one basic principle of pharmacological intervention (Evgenov et al. 2006; Stasch et al. 2009). Nitrates and PDE5 inhibitors (PDE5i) which could increase intra-cellular cGMP levels are therefore already approved therapies for Angina Pectoris and Pulmonary Hypertension (PAH) or Erectile Dysfunction (ED), respectively. sGC stimulators can overcome significant limitations of nitrates and PDE5i by direct stimulation of the soluble guanylate cyclase (sGC). sGC stimulators like Riociguat are approved for the treatment of Pulmonary Hypertension (PAH) and Chronic Thromboembolic Pulmonary Hypertension (CTEPH) or are in late stage Phase III clinical development for the treatment of Heart Failure (HFrEF). Moreover, additional sGC stimulators are in earlier stages of clinical development and preclinical investigation including e.g. Hypertension (HTN), Chronic Kidney Disease (CKD), Systemic Sclerosis (SSc), Cystic Fibrosis (CF), Sickle Cell Disease (SCD) and others. This very broad treatment potential of sGC stimulators underpins this very effective and broad pharmacological intervention strategy for various diseases. Therefore intense research efforts are still ongoing to understand the various modes of action of sGC stimulators to fully exploit the treatment potential to the benefit of patients.

It is well accepted that sGC stimulators act via direct stimulation of the sGC which does not require NO. The sGC stimulators bind to the non-oxidized and heme-containing sGC which leads to NO-independent formation and increase of intracellular cGMP (Stasch & Hobbs 2009). In addition, the sGC stimulators enhance the NO-effect on cGMP when NO is bound to the sGC. Therefore, sGC stimulators also exhibit synergistic effects with NO on cGMP production. The indazole derivative YC-1 was the first NO-independent but heme-dependent sGC stimulator described (Evgenov et al., 2006). Based on YC-1, further substances were discovered which are more potent than YC-1 and show no relevant inhibition of phosphodiesterases (PDE). This led to the identification of the pyrazolopyridine derivatives BAY 41-2272, BAY 41-8543 and BAY 63-2521 (Riociguat). Together with the structurally different substances CFM-1571 and A-350619, these compounds form the class of the sGC stimulators (Evgenov et al., 2006; Stasch and Hobbs, 2009). More recently further compound classes were discovered which show a different pharmacokinetic profile and also a different organ distribution which might have an impact on their treatment potential (Follmann et al. J. Med Chem 2017).

Under oxidative stress conditions, the Fe²⁺ iron atom of the heme group of the sGC is oxidized to Fe which destabilizes the binding of the heme group to the beta-subunit of the sGC and renders the enzyme heme-free. With the discovery of BAY 58-2667 (Cinaciguat) a new chemical matter has been found which is able to activate heme-free sGC. Therefore, BAY 58-2667 is the prototype of this class of sGC activators. Common characteristics of these substances are that in combination with NO they only have an additive effect on enzyme activation, and that the activation of the oxidized or heme-free enzyme is markedly higher than that of the heme-containing enzyme (Schmidt et al. 2009). Spectroscopic studies show that BAY 58-2667 displaces the oxidized heme group which, as a result of the weakening of the iron-histidine bond, is attached only weakly to the sGC. It has also been shown that the characteristic sGC heme binding motif Tyr-x-Ser-x-Arg is absolutely essential both for the interaction of the negatively charged propionic acids of the heme group and for the action of BAY 58-2667. Therefore, it is assumed that the binding site of BAY 58-2667 at the sGC is identical to the binding site of the heme group (Schmidt et al. 2009). More recently other classes of sGC activators were discovered which show a different pharmacokinetic profile and also a different organ distribution which might have an impact on their treatment potential.

It is well established that sGC stimulators and sGC activators lead to relaxation of vascular smooth muscle cells and blood pressure decrease. This is one of the basic principles for the use of sGC stimulators in cardiovascular diseases. However, other modes of action beyond vasodilation and targeting the vascular smooth muscle cells are only partly understood and are currently under investigation. In addition, it is also not well described in the art in which diseases and under which conditions and in which tissue or cell the increased oxidative stress leads to formation of heme-free sGC. However, hypoxic stimuli, especially in the brain, might cause formation of heme-free sGC in the central nervous system. It is difficult to predict the cellular effects and treatment effects of sGC stimulators and sGC activators since cGMP has multiple downstream targets, e.g. protein kinases, phosphodiesterases, ion channels, structural proteins, and potentially also unknown targets, which vary from cell to cell and from tissue to tissue and could also be substantially down- or upregulated in disease states. In line with this it became also obvious in recent years that a cGMP increase might have an impact on neuronal function and could be neuroprotective or might influence recognition and memory. WO 2003/095451 already mentions that sGC stimulators, including Riociguat, are also suitable for controlling central nervous system diseases characterized by disturbances of the NO/cGMP system, in particular for improving perception, concentration, learning or memory after cognitive impairments like those occurring in particular in association with situations/diseases/syndromes such as mild cognitive impairment, age-associated learning and memory impairments, age-associated memory loss, vascular dementia, Alzheimer's disease, Parkinson's disease, schizophrenia with dementia, and other diseases. Heckman et al. (2016 & 2018) reviewed clinical studies with regard to effects of different phosphodiesterase inhibitors on cognition, affect, and motor function in relation to the fronto-striatal circuits and concluded that PDE5 inhibitors have influence on striatal functions. However, Heckman et al. also point out that clinical trials investigating the effects of PDE-inhibitors in neuropsychiatric disorders are overall very sparse, and the wealth of positive preclinical data could not yet be translated into clinical efficacy. As a result, no definitive conclusions can be drawn merely based on these sparse clinical trial outcomes. K. Celikyurt et al. (2014) reported that chronic administration of the sGC stimulator YC-1 may affect age-related learning and memory dysfunction in aged rats. WO 2017/108441 A1 pertains to the treatment of cognitive impairment, in particular cognitive impairment associated with aging, Alzheimer's disease or schizophrenia, with the sGC stimulator Riociguat (BAY 63-2521) or its active metabolite Nelociguat (BAY 60-4552) in a mouse animal model. In particular, WO 2017/108441 A1 pertains to the treatment of cognitive impairment by administering Riociguat or Neliciguat in addition to an Acetylcholinesterase inhibitor. In WO 2017/108441 A1 it is concluded that Riociguat—at one single dose of 0.03 mg/kg—is able to enhance spatial memory in healthy mice. Higher (0.1-0.3 mg/kg) or lower (0.01 mg/kg) doses of Riociguat showed no significant effect in healthy mice. It is not possible to extrapolate from this single application of only one effective dose of riociguat in healthy mice to a chronic treatment regimen or even more difficult to a dose range which might be effective in patients with cognitive impairment.

According to the present invention, sGC activators or a pharmaceutically acceptable salt thereof and certain sGC stimulators, selected from methyl {4,6-diamino-2-[5-fluoro-1-(2-fluorobenzyl)-1H-pyrazolo[3,4-b]pyridine-3-yl]pyrimidin-5-yl}carbamate (Vericiguat, the compound of formula (5)) or a pharmaceutically acceptable salt thereof and ent-N-[(2S)-amino-2-methylbutyl]-8-[(2,6-difluorobenzyl)oxy]-2,6-dimethylimidazo[1,2-a]pyridine-3-carboxamide (enantiomer A) (the compound of formula (6)) or a pharmaceutically acceptable salt thereof improve cognitive function
at dosages which do not significantly reduce blood pressure
with a surprisingly broad therapeutic dose range compared to the state of the art (WO 2017/108441 A1)
at a dose that results in an overall exposure of the sGC activator or sGC stimulator which could not be expected to be effective according to the state of the art (WO 2017/108441 A1)

Surprisingly, sGC activators of formulae (1) and (2) or a pharmaceutically acceptable salt thereof and the sGC stimulators of formulae (5) and (6) or a pharmaceutically acceptable salt thereof could directly improve cognitive function. This was independent from blood pressure reduction. The surprisingly broad therapeutic range allows for better safety margins and dose adjustment.

The compounds described in the present invention are therefore effective for controlling diseases of the central nervous system with unexpected beneficial properties compared to the state of the art.

One embodiment of the invention is at least one sGC activator or a pharmaceutically acceptable salt thereof for use in the treatment of cognitive impairment in a mammal in need of such treatment.

As used herein, the term “activator” of soluble Guanylyl Cyclase (sGC) relates to an active compound that interacts with an oxidized or heme-free form of the sGC, to activate an oxidized or heme-free form of the sGC to catalyze the formation of cGMP (Schmidt et al. 2009).

As used herein, the term “activation” is to be understood as increasing the measured production of cGMP by at least 5% as compared to a control, e.g., a non-treated control, preferably by at least 10%, more preferably by at least 15%, even more preferably by at least 20%, even more preferably by at least 25%, even more preferably by at least 30% or by at least 40% or by at least 50%. Suitable controls are evident for the skilled person when considering the teaching of the present disclosure. Suitable assays to determine said activation are readily available to the skilled person from the pertinent literature. In one embodiment of the invention, assay A-3 is being used to determine said activation.

As used herein, the term “a dose is not significantly reducing blood pressure” is to be understood as a dose of a sGC activator or sGC stimulator compound according to the invention that does not reduce the blood pressure by more than 20% from baseline, preferably a dose that does not reduce the blood pressure by more than 15% from baseline, more preferably a dose a dose that does not reduce the blood pressure by more than 10% from baseline.

A further embodiment of the invention is at least one sGC activator or a pharmaceutically acceptable salt thereof for use in the treatment of cognitive impairment in a mammal in need of such treatment, wherein the cognitive impairment is selected from the group consisting of mild cognitive impairment, dementia, vascular dementia, Alzheimer dementia and cognitive impairment associated with cerebral infarctions, cerebral ischemia and ischemic stroke.

A further embodiment of the invention is at least one sGC activator or a pharmaceutically acceptable salt thereof for use in the treatment of cognitive impairment in a mammal in need of such treatment, wherein cognitive impairment is vascular dementia.

A further embodiment of the invention is at least one sGC activator or a pharmaceutically acceptable salt thereof for use in the treatment of cognitive impairment associated with cerebral infarctions, stroke, cerebral ischemia, ischemic stroke, head injury, post-stroke dementia, post-traumatic head injury, general disturbances of concentration, disturbances of concentration in children with learning, memory problems, Lewy body dementia, dementia with frontal lobe degeneration including Pick's syndrome, Parkinson's disease, progressive nuclear palsy, dementia with corticobasal degeneration, amyotrophic lateral sclerosis, Huntington's disease, demyelination, multiple sclerosis, thalamic degeneration, Creutzfeldt-Jakob dementia, HIV-dementia, schizophrenia or Korsakoff psychosis.

According to a further embodiment of the present invention, the sGC activator for use according to the invention is selected from the group consisting of:
4-({(4-carboxybutyl) [2-(2-{[4-(2-phenylethyl)benzyl]oxy}phenyl)ethyl]amino}methyl)benzoic acid
4-({(4-carboxybutyl)[2-(5-fluoro-2-{[4′-(trifluoromethyl)[1,1′-biphenyl]-4-yl]methoxy}phenyl)ethyl]amino}methyl)benzoic acid (compound of formula (2), BAY 60-2770)
5-chloro-2-(5-chlorothiophene-2-sulfonylamino-N-(4-(morpholine-4-sulfonyl)phenyl)benzamide as sodium salt
2-(4-chlorophenylsulfonylamino)-4,5-dimethoxy-N-(4-(thiomorpholine-4-sulfonyl)phenyl)benzamide
1-{6-[5-chloro-2-({4-trans-4-}trifluoromethyl)cyclohexyl]benzyl}oxy)phenyl]pyridin-2-yl}-5-(trifluoromethyl)-1H-pyrazole-4-carboxylic acid
1-[6-(2-(2-methyl-4-(4-trifluoromethoxyphenyl)benzyloxy)phenyl)pyridin-2-yl]-5-trifluoromethylpyrazole-4-carboxylic acid
1[6-(3,4-dichlorophenyl)-2-pyridinyl-5-(trifluoromethyl)-1H-pyrazole-4-carboxylic acid
1-({2-[3-chloro-5-(trifluoromethyl)phenyl]-5-methyl-1,3-thiazol-4-yl}methyl)-1H-pyrazole-4-carboxylic acid
4-({2-[3-(trifluoromethyl)phenyl]-1,3-thiazol-4-yl}methyl)benzoic acid
1-({2-[2-fluoro-3-(trifluoromethyl)phenyl]-5-methyl-1,3-thiazol-4-yl}methyl)-1H-pyrazole-4-carboxylic acid
3-(4-chloro-3-{[(2S,3R)-2-(4-chlorophenyl)-4,4,4-trifluoro-3-methylbutanoyl]amino}phenyl)-3-cyclopropylpropanoic acid (compound of formula (1)), known from WO 2012/139888, example 22
5-{[2-(4-carboxyphenyl)ethyl][2-(2-{[3-chloro-4′-(trifluoromethyl)biphenyl-4-yl]methoxy}phenyl)ethyl]amino}-5,6,7,8-tetrahydroquinoline-2-carboxylic acid (compound of formula (4)), known from WO 2014/012934, example 23
5-{(4-carboxybutyl)[2-(2-{[3-chloro-4′-(trifluoromethyl)biphenyl-4-yl]methoxy}phenyl)ethyl]amino}-5,6,7,8-tetrahydroquinoline-2-carboxylic acid (compound of formula (3)), known from WO 2014/012934, example 7
(1R,5S)-3-[4-(5-methyl-2-{[2-methyl-4-(piperidin-1-ylcarbonyl)benzyl]oxy}phenyl)-1,3-thiazol-2-yl]-3-azabicyclo[3.2.1]octane-8-carboxylic acid and
1-[6-(5-methyl-2-{[2-(tetrahydro-2H-pyran-4-yl)-1,2,3,4-tetrahydroisoquinolin-6-yl]methoxy}phenyl)pyridin-2-yl]-5-(trifluoromethyl)-1H-pyrazole-4-carboxylic acid,
   or a pharmaceutically acceptable salt thereof.

According to a further embodiment of the present invention, the sGC activator for use according to the invention is selected from the group consisting of:
3-(4-chloro-3-{[(2S,3R)-2-(4-chlorophenyl)-4,4,4-trifluoro-3-methylbutanoyl]amino}phenyl)-3-cyclopropylpropanoic acid (compound of formula (1))
4-({(4-carboxybutyl)[2-(5-fluoro-2-{[4′-(trifluoromethyl)[1,1′-biphenyl]-4-yl]methoxy}phenyl)ethyl]amino}methyl)benzoic acid (compound of formula (2))
5-{(4-carboxybutyl)[2-(2-{[3-chloro-4′-(trifluoromethyl)biphenyl-4-yl]methoxy}phenyl)ethyl]amino}-5,6,7,8-tetrahydroquinoline-2-carboxylic acid (compound of formula (3)) and
5-{[2-(4-carboxyphenyl)ethyl][2-(2-{[3-chloro-4′-(trifluoromethyl)biphenyl-4-yl]methoxy}phenyl)ethyl]amino}-5,6,7,8-tetrahydroquinoline-2-carboxylic acid (compound of formula (4)),

or a pharmaceutically acceptable salt thereof.

According to a further embodiment of the present invention, the sGC activator for use according to the invention is:
3-(4-chloro-3-{[(2S,3R)-2-(4-chlorophenyl)-4,4,4-trifluoro-3-methylbutanoyl]amino}phenyl)-3-cyclopropylpropanoic acid (compound of formula (1))

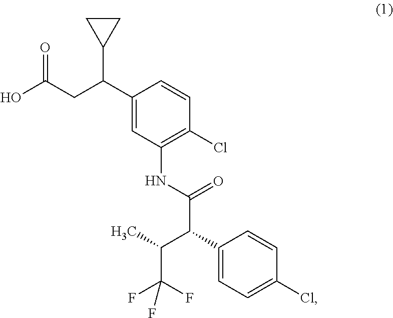 [SMILES: C[C@H]([C@H](C(=O)Nc1cc(C(CC(=O)O)C2CC2)ccc1Cl)c1ccc(Cl)cc1)C(F)(F)F]

or a pharmaceutically acceptable salt thereof.

According to a further embodiment of the present invention, the sGC activator for use according to the invention is:
4-({(4-carboxybutyl)[2-(5-fluoro-2-{[4′-(trifluoromethyl)[1,1′-biphenyl]-4-yl]methoxy}phenyl)ethyl]amino}methyl)benzoic acid (compound of formula (2))

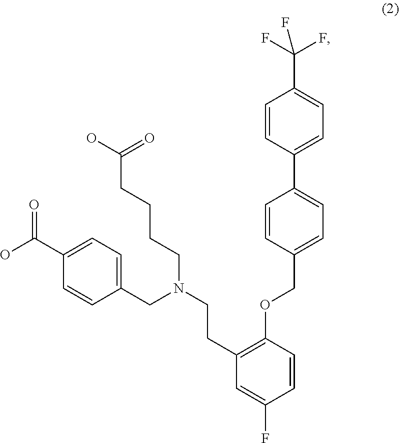 [SMILES: O=C(O)CCCCN(CCc1cc(F)ccc1OCc1ccc(-c2ccc(C(F)(F)F)cc2)cc1)Cc1ccc(C(=O)O)cc1]

or a pharmaceutically acceptable salt thereof.

A further embodiment of the invention is at least one sGC activator or a pharmaceutically acceptable salt thereof for use in the treatment of cognitive impairment in a mammal in need of such treatment, wherein the at least one sGC activator is administered at a daily dose of 0.2 to 25 mg.

A further embodiment of the invention is at least one sGC activator or a pharmaceutically acceptable salt thereof for use in the treatment of cognitive impairment in a mammal in need of such treatment, wherein the at least one sGC activator is administered at a daily dose of 0.5 to 25 mg.

A further embodiment of the invention is at least one sGC activator or a pharmaceutically acceptable salt thereof for use in the treatment of cognitive impairment in a mammal in need of such treatment, wherein the at least one sGC activator is administered at a daily dose of 0.5 to 10 mg.

A further embodiment of the invention is at least one sGC activator or a pharmaceutically acceptable salt thereof for use in the treatment of cognitive impairment in a mammal in need of such treatment, wherein the at least one sGC activator is administered at a daily dose of 1 to 10 mg.

A further embodiment of the invention is at least one sGC activator selected from the group consisting of the compounds of formulae (1) to (4)
3-(4-chloro-3-{[(2S,3R)-2-(4-chlorophenyl)-4,4,4-trifluoro-3-methylbutanoyl]amino}phenyl)-3-cyclopropylpropanoic acid of formula (1),
4-({(4-carboxybutyl)[2-(5-fluoro-2-{[4′-(trifluoromethyl)[1,1′-biphenyl]-4-yl]methoxy}phenyl)ethyl]amino}methyl)benzoic acid of formula (2),
5-{(4-carboxybutyl)[2-(2-{1[3-chloro-4′-(trifluoromethyl)biphenyl-4-yl]methoxy}phenyl)ethyl]amino}-5,6,7,8-tetrahydroquinoline-2-carboxylic acid of formula (3), and
5-{[2-(4-carboxyphenyl)ethyl][2-(2-{[3-chloro-4′-(trifluoromethyl)biphenyl-4-yl]methoxy}phenyl)ethyl]amino}-5,6,7,8-tetrahydroquinoline-2-carboxylic acid of formula (4)

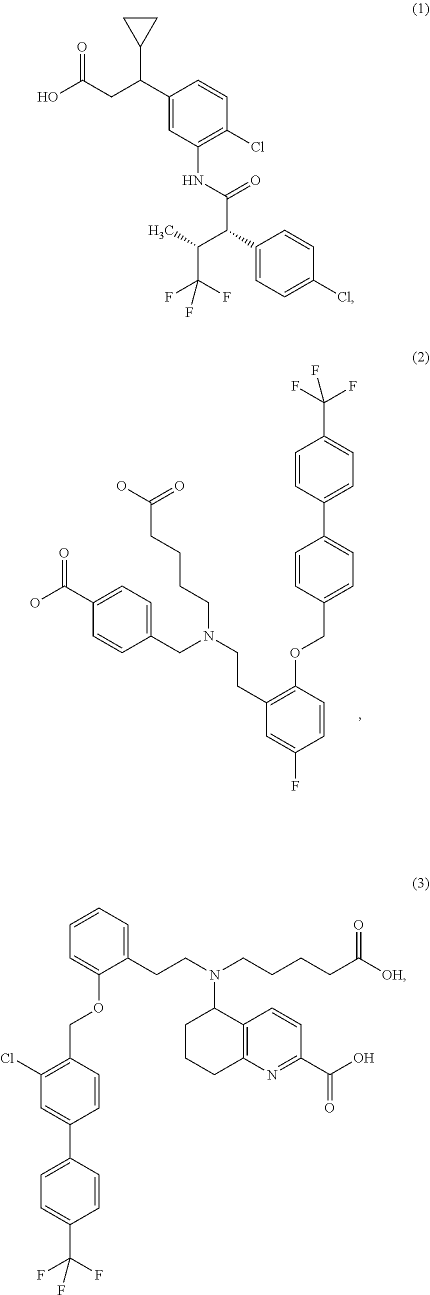

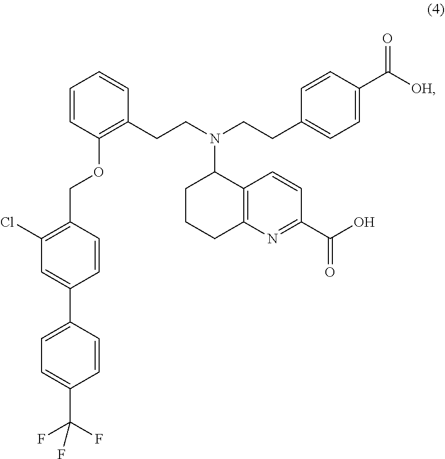 [SMILES: O=C(O)c1ccc(CCN(CCc2ccccc2OCc2ccc(-c3ccc(C(F)(F)F)cc3)cc2Cl)C2CCCc3nc(C(=O)O)ccc32)cc1]

or a pharmaceutically acceptable salt thereof for use in the treatment of cognitive impairment in a mammal in need of such treatment, wherein the at least one sGC activator is administered at a daily dose of 0.2 to 25 mg.

A further embodiment of the invention is at least one sGC activator selected from the compounds of formulae (1) to (4) or a pharmaceutically acceptable salt thereof for use in the treatment of cognitive impairment in a mammal in need of such treatment, wherein the at least one sGC activator is administered at a daily dose of 0.5 to 25 mg.

A further embodiment of the invention is at least one sGC activator selected from the compounds of formulae (1) to (4) or a pharmaceutically acceptable salt thereof for use in the treatment of cognitive impairment in a mammal in need of such treatment, wherein the at least one sGC activator is administered at a daily dose of 0.5 to 10 mg.

A further embodiment of the invention is at least one sGC activator selected from the compounds of formulae (1) to (4) or a pharmaceutically acceptable salt thereof for use in the treatment of cognitive impairment in a mammal in need of such treatment, wherein the at least one sGC activator is administered at a daily dose of 1 to 10 mg.

A further embodiment of the invention is the sGC activator of formula (1) or a pharmaceutically acceptable salt thereof for use in the treatment of cognitive impairment in a mammal in need of such treatment, wherein the at least one sGC activator is administered at a daily dose of 0.2 to 25 mg.

A further embodiment of the invention is the sGC activator of formula (1) or a pharmaceutically acceptable salt thereof for use in the treatment of cognitive impairment in a mammal in need of such treatment, wherein the at least one sGC activator is administered orally at a daily dose of 0.2 to 25 mg.

A further embodiment of the invention is the sGC activator of formula (1) or a pharmaceutically acceptable salt thereof for use in the treatment of cognitive impairment in a mammal in need of such treatment, wherein the at least one sGC activator is administered at a daily dose of 0.3 to 10 mg.

A further embodiment of the invention is the sGC activator of formula (1) or a pharmaceutically acceptable salt thereof for use in the treatment of cognitive impairment in a mammal in need of such treatment, wherein the at least one sGC activator is administered orally at a daily dose of 1 to 10 mg.

A further embodiment of the invention is the sGC activator of formula (2) or a pharmaceutically acceptable salt thereof for use in the treatment of cognitive impairment in a mammal in need of such treatment, wherein the at least one sGC activator is administered at a daily dose of 0.2 to 25 mg.

A further embodiment of the invention is the sGC activator of formula (2) or a pharmaceutically acceptable salt thereof for use in the treatment of cognitive impairment in a mammal in need of such treatment, wherein the at least one sGC activator is administered at a daily dose of 0.5 to 10 mg.

A further embodiment of the invention is at least one sGC activator selected from the compounds of formulae (1) to (4) or a pharmaceutically acceptable salt thereof for use in the treatment of cognitive impairment in a mammal in need of such treatment, wherein the orally administered daily dose is not significantly reducing blood pressure.

A further embodiment of the invention is the sGC activator of formula (1) or a pharmaceutically acceptable salt thereof for use in the treatment of cognitive impairment in a mammal in need of such treatment, wherein the orally administered daily dose is not significantly reducing blood pressure.

A further embodiment of the invention is the sGC activator of formula (1) or a pharmaceutically acceptable salt thereof for use in the treatment of cognitive impairment in a mammal in need of such treatment, wherein the sGC activator of formula (1) administered orally at a daily dose of 0.2 to 25 mg or 0.3 to 10 mg or 1 to 10 mg and this dose is not significantly reducing blood pressure.

A further embodiment of the invention is the sGC activator of formula (2) or a pharmaceutically acceptable salt thereof for use in the treatment of cognitive impairment in a mammal in need of such treatment, wherein the orally administered daily dose is not significantly reducing blood pressure.

Further sGC activators in the context of the invention are known from the following publications: WO2013/157528, WO2015/056663, WO2009/123316, WO2016/001875, WO2016/001876, WO2016/001878, WO2000/02851, WO2012/122340, WO2013/025425, WO2014/039434, WO2016/014463, WO2009/068652, WO2009/071504, WO2010/015652, WO2010/015653, WO2015/033307, WO2016/042536, WO2009/032249, WO2010/099054, WO2012/058132, US2010/0216764, WO2001/19776, WO2001/19780, WO2001/19778, WO2002/070459, WO2002/070460, WO2002/070510, WO2002/070462, WO2007/045366, WO2007/045369, WO2007/045433, WO2007/045370, WO2007/045367, WO2014/012935, WO2014/012934, WO2011/141409, WO2008/119457, WO2008/119458, WO2009/127338, WO2010/102717, WO2011/051165, WO2012/076466, WO2012/139888, WO2013/157528, WO2013/174736, WO2014/012934, WO2015/056663, WO2017103888, WO2017112617, WO2016042536, WO2016081668, WO2016191335, WO2016191334, WO2016001875, WO2016001876, WO2016001878, WO2016014463, WO2016044447, WO2016044445, WO2016044446, WO2015056663, WO2015033307, WO2015187470, WO2015088885, WO2015088886, WO2015089182, WO2014084312, WO2014039434, WO2014144100, WO2014047111, WO2014047325, WO2013025425, WO2013101830, WO2012165399, WO2012058132, WO2012122340, WO2012003405, WO2012064559, WO2011149921, WO2011119518, WO2011115804, WO2011056511, CN101670106, TW201028152, WO2010015653, WO2010015652, WO2010099054, WO2010065275, WO2009123316, WO2009068652, WO2009071504, WO2009032249, US2009209556.

One embodiment of the invention is at least one sGC stimulator for use in the treatment of cognitive impairment in a mammal in need of such treatment, wherein the at least on sGC stimulator is selected from the group consisting of:
2-[1-(2-fluorobenzyl)-1H-pyrazolo[3,4-b]pyridin-3-yl]-5-(4-morpholinyl)-4,6-pyrimidinediamine
2-[1-(2-fluorobenzyl)-1H-pyrazolo[3,4-b]pyridin-3-yl]-5-(4-pyridinyl)-4-pyrimidineamine
methyl{4,6-diamino-2-[5-fluoro-1-(2-fluorobenzyl)-1H-pyrazolo[3,4-b]pyridine-3-yl]pyrimidin-5-yl}carbamate (Vericiguat, compound of formula (5)), known from WO 2011/147809, example 1
methyl{4,6-diamino-2-[5-fluoro-1-(2-fluorobenzyl)-1H-pyrazolo[3,4-b]pyridine-3-yl]pyrimidin-5-yl}methylcarbamate
methyl{4,6-diamino-2-[5-fluoro-1-(2-fluorobenzyl)-1H-pyrazolo[3,4-b]pyridin-3-yl]pyrimidin-5-yl}(2,2,2-trifluoroethyl)carbamate
4-amino-2-[5-chloro-3(3,3,3-trifluoropropyl)-1H-indazol-1-yl]-5,5-dimethyl-5,7-dihydro-6H-pyrrolo[2,3-d]pyrimidin-6-one
4-amino-2[5-chloro-3-(2,3,6-trifluorobenzyl)-1H-indazol-1-yl]-5,5-dimethyl-5,7-dihydro-6H-pyrrolo[2,3-d]pyrimidin-6-one
4-amino-5,5-dimethyl-2-[3-(2,3,6-trifluorobenzyl)1H-thieno[3,4-c]pyrazol-1-yl]-5,7-dihydro-6H-pyrrolo[2,3-d]pyrimidin-6-one
4-amino-5,5-dimethyl-2-[3-(2,3,6-trifluorobenzyl)-1H-thieno[2,3-d]pyrazol-1-yl]-5,5-dimethyl-5,7-dihydro-6H-pyrrolo[2,3-d]pyrimidin-6-one
4-amino-5,5-dimethyl-2-[7-(2,3,6-trifluorobenzyl)imidazo[1,5-b]pyridazin-5-yl]-5,7-dihydro-6H-pyrrolo[2,3-d]pyrimidin-6-one
4-amino-2-[6-chloro-3-(2,3,6-trifluorobenzyl)imidazo[1,5-a]pyridin-1-yl]]-5,5-dimethyl-5,7-dihydro-6H-pyrrolo[2,3-d]pyrimidin-6-one
4-amino-2-[6-fluoro-3-(2,3,6-trifluorobenzyl)imidazo[1,5-a]pyridin-1-yl]]-5,5-dimethyl-5,7-dihydro-6H-pyrrolo[2,3-d]pyrimidin-6-one
4-amino-2-[6-fluoro-3-(2,3,6-trifluorobenzyl)6-fluoroimidazo[1,5-a]pyridin-1-yl]-5,5-dimethyl-5,7-dihydro-6H-pyrrolo[2,3-d]pyrimidin-6-one
4-amino-5,5-dimethyl-2-[3-(2,4,6-trifluorobenzyl)imidazo[1,5-a]pyridin-1-yl]]-5,7-dihydro-6H-pyrrolo[2,3-d]pyrimidin-6-one
4-amino-2-[3-(2-cyclopentylethyl)imidazo[1,5-a]pyridin-1-yl]-5,5-dimethyl-5,7-dihydro-6H-pyrrolo[2,3-d]pyrimidin-6-one
3-(4-amino-5-cyclopropylpyrimidin-2-yl)-1-(2-fluorobenzyl)-1H-pyrazolo[3,4-b]pyridine
2-{5-fluoro-1-[(3-fluoropyridin-2-yl)methyl]-1H-pyrazolo[3,4-b]pyridin-3-yl}-5-methyl-5-(trifluoromethyl)-4-[(3,3,3-trifluoropropyl)amino]-5,7-dihydro-6H-pyrrolo[2,3-d]pyrimidin-6-one
ent-N-[(2S)-amino-2-methylbutyl]-8-[(2,6-difluorobenzyl)oxy]-2,6-dimethylimidazo[1,2-a]pyridine-3-carboxamide (enantiomer A, compound of formula (6)), known from WO 2014/068099, example 200
ent-N-(2-amino-2-methylbutyl)-8-[(2,6-difluorobenzyl)oxy]-2,6-dimethylimidazo[1,2-a]pyridine-3-carboxamide (enantiomer B)
ent-N-(2-amino-5,5,5-trifluoro-2-methylpentyl)-2,6-dimethyl-8-[(2,3,6-trifluorobenzyl)oxy]imidazo[1,2-a]pyridine-3-carboxamide (enantiomer B)
ent-N-(2-amino-5,5,5-trifluoro-2-methylpentyl)-8-[(2,6-difluorobenzyl)oxy]-2,6-dimethylimidazo[1,2-a]pyridine-3-carboxamide (enantiomer B)
ent-N-(2-amino-5,5,5-trifluoro-2-methylpentyl)-8-[(2,6-difluorobenzyl)oxy]-2,6-dimethylimidazo[1,2-a]pyridine-3-carboxamide (enantiomer A)
ent-N-(2-amino-3-fluoro-2-methylpropyl)-2,6-dimethyl-8-[(2,3,6-trifluorobenzyl)oxy]imidazo[1,2-a]pyridine-3-carboxamide (enantiomer B)
ent-N-(2-amino-3-fluoro-2-methylpropyl)-8-[(2,6-difluorobenzyl)oxy]-2,6-dimethylimidazo[1,2-a]pyridine-3-carboxamide (enantiomer B)
ent-N-(2-amino-3-fluoro-2-methylpropyl)-8-[(2,6-difluorobenzyl)oxy]-2,6-dimethylimidazo[1,2-a]pyridine-3-carboxamide (enantiomer A)
rac-N-(2-amino-3-fluoro-2-methylpropyl)-8-[(2,6-difluorobenzyl)oxy]-2,6-dimethylimidazo[1,2-a]pyridine-3-carboxamide formate
ent-N-(2-amino-3-fluoro-2-methylpropyl)-2,6-dimethyl-8-[(2,3,6-trifluorobenzyl)oxy]imidazo[1,2-a]pyridine-3-carboxamide (enantiomer A)
ent-N-(2-amino-3-fluoro-2-methylpropyl)-8-[(2,6-difluorobenzyl)oxy]-6-(difluoromethyl)-2-methylimidazo[1,2-a]pyridine-3-carboxamide (enantiomer B)
ent-N-(2-amino-3-fluoro-2-methylpropyl)-8-[(2,6-difluorobenzyl)oxy]-6-(difluoromethyl)-2-methylimidazo[1,2-a]pyridine-3-carboxamide (enantiomer A)
ent-N-(2-amino-3-fluoro-2-methylpropyl)-8-[(2,6-difluorobenzyl)oxy]-6-(fluoromethyl)-2-methylimidazo[1,2-a]pyridine-3-carboxamide
1,1,1,3,3,3-Hexafluoro-2-[({5-fluoro-2-[1-(2-fluorobenzyl)-5-(1,2-oxazol-3-yl)-1H-pyrazol-3-yl]-4-pyrimidinyl}amino)methyl]-2-propanol (Praliciguat)
5-fluoro-2-[1-(2-fluorobenzyl)-5-(1,2-oxazol-3-yl)-1H-pyrazol-3-yl]pyrimidin-4-ol (IWP-051)
IWP-121, IWP-427, IWP-953, IW-1701, and IW-6463,

or a pharmaceutically acceptable salt thereof.

According to a further embodiment of the present invention, sGC stimulators for use according to the invention are selected from the group consisting of:
2-[1-(2-fluorobenzyl)-1H-pyrazolo[3,4-b]pyridin-3-yl]-5-(4-morpholinyl)-4,6-pyrimidinediamine
2-[1-(2-fluorobenzyl)-1H-pyrazolo[3,4-b]pyridin-3-yl]-5-(4-pyridinyl)-4-pyrimidineamine
methyl{4,6-diamino-2-[5-fluoro-1-(2-fluorobenzyl)-1H-pyrazolo[3,4-b]pyridine-3-yl]pyrimidin-5-yl}carbamate (Vericiguat, compound of formula (5))
ent-N-[(2S)-amino-2-methylbutyl]-8-[(2,6-difluorobenzyl)oxy]-2,6-dimethylimidazo[1,2-a]pyridine-3-carboxamide (enantiomer A, compound of formula (6)).
ent-N-(2-amino-2-methylbutyl)-8-[(2,6-difluorobenzyl)oxy]-2,6-dimethylimidazo[1,2-a]pyridine-3-carboxamide (enantiomer B)
methyl{4,6-diamino-2-[5-fluoro-1-(2-fluorobenzyl)-1H-pyrazolo[3,4-b]pyridine-3-yl]pyrimidin-5-yl}methylcarbamate
methyl{4,6-diamino-2-[5-fluoro-1-(2-fluorobenzyl)-1H-pyrazolo[3,4-b]pyridin-3-yl]pyrimidin-5-yl}(2,2,2-trifluoroethyl)carbamate
4-amino-2-[5-chloro-3(3,3,3-trifluoropropyl)-1H-indazol-1-yl]-5,5-dimethyl-5,7-dihydro-6H-pyrrolo[2,3-d]pyrimidin-6-one
4-amino-2[5-chloro-3-(2,3,6-trifluorobenzyl)-1H-indazol-1-yl]-5,5-dimethyl-5,7-dihydro-6H-pyrrolo[2,3-d]pyrimidin-6-one
4-amino-5,5-dimethyl-2-[3-(2,3,6-trifluorobenzyl)1H-thieno[3,4-c]pyrazol-1-yl]-5,7-dihydro-6H-pyrrolo[2,3-d]pyrimidin-6-one
4-amino-5,5-dimethyl-2-[3-(2,3,6-trifluorobenzyl)-1H-thieno[2,3-d]pyrazol-1-yl]-5,5-dimethyl-5,7-dihydro-6H-pyrrolo[2,3-d]pyrimidin-6-one
4-amino-5,5-dimethyl-2-[7-(2,3,6-trifluorobenzyl)imidazo[1,5-b]pyridazin-5-yl]-5,7-dihydro-6H-pyrrolo[2,3-d]pyrimidin-6-one
4-amino-2-[6-chloro-3-(2,3,6-trifluorobenzyl)imidazo[1,5-a]pyridin-1-yl]]-5,5-dimethyl-5,7-dihydro-6H-pyrrolo[2,3-d]pyrimidin-6-one
4-amino-2-[6-fluoro-3-(2,3,6-trifluorobenzyl)imidazo[1,5-a]pyridin-1-yl]]-5,5-dimethyl-5,7-dihydro-6H-pyrrolo[2,3-d]pyrimidin-6-one
4-amino-2-[6-fluoro-3-(2,3,6-trifluorobenzyl)6-fluoroimidazo[1,5-a]pyridin-1-yl]-5,5-dimethyl-5,7-dihydro-6H-pyrrolo[2,3-d]pyrimidin-6-one
4-amino-5,5-dimethyl-2-[3-(2,4,6-trifluorobenzyl)imidazo[1,5-a]pyridin-1-yl]]-5,7-dihydro-6H-pyrrolo[2,3-d]pyrimidin-6-one
4-amino-2-[3-(2-cyclopentylethyl)imidazo[1,5-a]pyridin-1-yl]-5,5-dimethyl-5,7-dihydro-6H-pyrrolo[2,3-d]pyrimidin-6-one
3-(4-amino-5-cyclopropylpyrimidin-2-yl)-1-(2-fluorobenzyl)-1H-pyrazolo[3,4-b]pyridine
2-{5-fluoro-1-[(3-fluoropyridin-2-yl)methyl]-1H-pyrazolo[3,4-b]pyridin-3-yl}-5-methyl-5-(trifluoromethyl)-4-[(3,3,3-trifluoropropyl)amino]-5,7-dihydro-6H-pyrrolo[2,3-d]pyrimidin-6-one and
1,1,1,3,3,3-Hexafluoro-2-[({5-fluoro-2-[1-(2-fluorobenzyl)-5-(1,2-oxazol-3-yl)-1H-pyrazol-3-yl]-4-pyrimidinyl}amino)methyl]-2-propanol (Praliciguat),

or a pharmaceutically acceptable salt thereof.

According to a further embodiment of the present invention, sGC stimulators for use according to the invention are selected from the group consisting of:
2-[1-(2-fluorobenzyl)-1H-pyrazolo[3,4-b]pyridin-3-yl]-5-(4-morpholinyl)-4,6-pyrimidinediamine
2-[1-(2-fluorobenzyl)-1H-pyrazolo[3,4-b]pyridin-3-yl]-5-(4-pyridinyl)-4-pyrimidineamine
methyl{4,6-diamino-2-[5-fluoro-1-(2-fluorobenzyl)-1H-pyrazolo[3,4-b]pyridine-3-yl]pyrimidin-5-yl}carbamate (Vericiguat, compound of formula (5))
ent-N-[(2S)-amino-2-methylbutyl]-8-[(2,6-difluorobenzyl)oxy]-2,6-dimethylimidazo[1,2-a]pyridine-3-carboxamide (enantiomer A, compound of formula (6))
ent-N-(2-amino-2-methylbutyl)-8-[(2,6-difluorobenzyl)oxy]-2,6-dimethylimidazo[1,2-a]pyridine-3-carboxamide (enantiomer B)
methyl{4,6-diamino-2-[5-fluoro-1-(2-fluorobenzyl)-1H-pyrazolo[3,4-b]pyridine-3-yl]pyrimidin-5-yl}methylcarbamate
3-(4-amino-5-cyclopropylpyrimidin-2-yl)-1-(2-fluorobenzyl)-1H-pyrazolo[3,4-b]pyridine
2-{5-fluoro-1-[(3-fluoropyridin-2-yl)methyl]-1H-pyrazolo[3,4-b]pyridin-3-yl}-5-methyl-5-(trifluoromethyl)-4-[(3,3,3-trifluoropropyl)amino]-5,7-dihydro-6H-pyrrolo[2,3-d]pyrimidin-6-one and
1,1,1,3,3,3-Hexafluoro-2-[({5-fluoro-2-[1-(2-fluorobenzyl)-5-(1,2-oxazol-3-yl)-1H-pyrazol-3-yl]-4-pyrimidinyl}amino)methyl]-2-propanol (Praliciguat), or a pharmaceutically acceptable salt thereof.

According to a further embodiment of the present invention, sGC stimulators for use according to the invention are selected from the group consisting of:
methyl{4,6-diamino-2-[5-fluoro-1-(2-fluorobenzyl)-1H-pyrazolo[3,4-b]pyridine-3-yl]pyrimidin-5-yl}carbamate (Vericiguat, compound of formula (5))
ent-N-[(2S)-amino-2-methylbutyl]-8-[(2,6-difluorobenzyl)oxy]-2,6-dimethylimidazo[1,2-a]pyridine-3-carboxamide (enantiomer A, compound of formula (6)).
ent-N-(2-amino-2-methylbutyl)-8-[(2,6-difluorobenzyl)oxy]-2,6-dimethylimidazo[1,2-a]pyridine-3-carboxamide (enantiomer B)
methyl{4,6-diamino-2-[5-fluoro-1-(2-fluorobenzyl)-1H-pyrazolo[3,4-b]pyridine-3-yl]pyrimidin-5-yl}methylcarbamate
2-{5-fluoro-1-[(3-fluoropyridin-2-yl)methyl]-1H-pyrazolo[3,4-b]pyridin-3-yl}-5-methyl-5-(trifluoromethyl)-4-[(3,3,3-trifluoropropyl)amino]-5,7-dihydro-6H-pyrrolo[2,3-d]pyrimidin-6-one and
1,1,1,3,3,3-Hexafluoro-2-[({5-fluoro-2-[1-(2-fluorobenzyl)-5-(1,2-oxazol-3-yl)-1H-pyrazol-3-yl]-4-pyrimidinyl}amino)methyl]-2-propanol (Praliciguat),

or a pharmaceutically acceptable salt thereof.

According to a further embodiment of the present invention, sGC stimulators for use according to the invention are selected from the group consisting of:
methyl{4,6-diamino-2-[5-fluoro-1-(2-fluorobenzyl)-1H-pyrazolo[3,4-b]pyridine-3-yl]pyrimidin-5-yl}carbamate (Vericiguat, compound of formula (5))
ent-N-[(2S)-amino-2-methylbutyl]-8-[(2,6-difluorobenzyl)oxy]-2,6-dimethylimidazo[1,2-a]pyridine-3-carboxamide (enantiomer A, compound of formula (6)).
ent-N-(2-amino-2-methylbutyl)-8-[(2,6-difluorobenzyl)oxy]-2,6-dimethylimidazo[1,2-a]pyridine-3-carboxamide (enantiomer B) and
1,1,1,3,3,3-Hexafluoro-2-[({5-fluoro-2-[1-(2-fluorobenzyl)-5-(1,2-oxazol-3-yl)-1H-pyrazol-3-yl]-4-pyrimidinyl}amino)methyl]-2-propanol (Praliciguat),

or a pharmaceutically acceptable salt thereof.

According to a further embodiment of the present invention, the sGC stimulator for use according to the invention is:
methyl{4,6-diamino-2-[5-fluoro-1-(2-fluorobenzyl)-1H-pyrazolo[3,4-b]pyridine-3-yl]pyrimidin-5-yl}carbamate (Vericiguat, compound of formula (5))

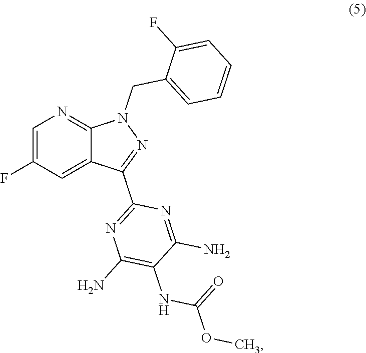 [SMILES: COC(=O)Nc1c(N)nc(-c2nn(Cc3ccccc3F)c3ncc(F)cc23)nc1N]

or a pharmaceutically acceptable salt thereof.

According to a further embodiment of the present invention, the sGC stimulator for use according to the invention is:
ent-N-[(2S)-amino-2-methylbutyl]-8-[(2,6-difluorobenzyl)oxy]-2,6-dimethylimidazo[1,2-a]pyridine-3-carboxamide (enantiomer A) (compound of formula (6))

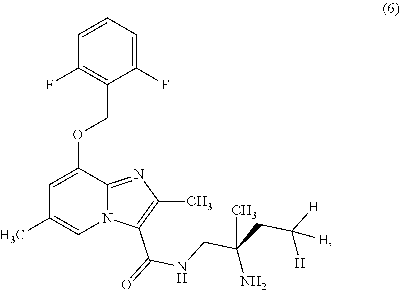

or a pharmaceutically acceptable salt thereof.

A further embodiment of the invention is at least one sGC stimulator selected from one of the groups specified above or a pharmaceutically acceptable salt thereof for use in the treatment of cognitive impairment in a mammal in need of such treatment, wherein the at least one sGC stimulator or a pharmaceutically acceptable salt thereof is selected from one of the groups specified above is administered at a daily dose of 0.2 to 25 mg.

A further embodiment of the invention is at least one sGC stimulator selected from the compounds of formulae (5) and (6) or a pharmaceutically acceptable salt thereof for use in the treatment of cognitive impairment in a mammal in need of such treatment, wherein the at least one compound of formulae (5) and (6) or a pharmaceutically acceptable salt thereof is administered at a daily dose of 0.2 to 25 mg.

A further embodiment of the invention is at least one sGC stimulator selected from the compounds of formulae (5) and (6) or a pharmaceutically acceptable salt thereof for use in the treatment of cognitive impairment in a mammal in need of such treatment, wherein the orally administered daily dose is not significantly reducing blood pressure.

A further embodiment of the invention is the compound of formula (5) or a pharmaceutically acceptable salt thereof for use in the treatment of cognitive impairment in a mammal in need of such treatment, wherein the compound of formula (5) or a pharmaceutically acceptable salt thereof is administered at a daily dose of 0.5 to 25 mg.

A further embodiment of the invention is the compound of formula (5) or a pharmaceutically acceptable salt thereof for use in the treatment of cognitive impairment in a mammal in need of such treatment, wherein the compound of formula (5) or a pharmaceutically acceptable salt thereof is administered at a daily dose of 1 to 10 mg.

A further embodiment of the invention is the compound of formula (5) or a pharmaceutically acceptable salt thereof for use in the treatment of cognitive impairment in a mammal in need of such treatment, wherein the compound of formula (5) or a pharmaceutically acceptable salt thereof is administered orally at a daily dose of 1 to 6 mg.

A further embodiment of the invention is the compound of formula (5) or a pharmaceutically acceptable salt thereof for use in the treatment of cognitive impairment in a mammal in need of such treatment, wherein the compound of formula (5) or a pharmaceutically acceptable salt thereof is administered orally at a daily dose of 1 to 3 mg.

A further embodiment of the invention is the compound of formula (5) or a pharmaceutically acceptable salt thereof for use in the treatment of cognitive impairment in a mammal in need of such treatment, wherein the orally administered daily dose is not significantly reducing blood pressure.

A further embodiment of the invention is the compound of formula (5) or a pharmaceutically acceptable salt thereof for use in the treatment of cognitive impairment in a mammal in need of such treatment, wherein the compound of formula (1) administered orally at a daily dose of 1 to 6 mg or 1 to 3 mg and this dose is not significantly reducing blood pressure.

A further embodiment of the invention is the compound of formula (6) or a pharmaceutically acceptable salt thereof for use in the treatment of cognitive impairment in a mammal in need of such treatment, wherein the compound of formula (6) or a pharmaceutically acceptable salt thereof is administered at a daily dose of 0.2 to 25 mg.

A further embodiment of the invention is the compound of formula (6) or a pharmaceutically acceptable salt thereof for use in the treatment of cognitive impairment in a mammal in need of such treatment, wherein the compound of formula (6) or a pharmaceutically acceptable salt thereof is administered at a daily dose of 0.3 to 10 mg.

A further embodiment of the invention is the compound of formula (6) or a pharmaceutically acceptable salt thereof for use in the treatment of cognitive impairment in a mammal in need of such treatment, wherein the compound of formula (6) or a pharmaceutically acceptable salt thereof is administered orally at a daily dose of 0.2 to 6 mg.

A further embodiment of the invention is the compound of formula (6) or a pharmaceutically acceptable salt thereof for use in the treatment of cognitive impairment in a mammal in need of such treatment, wherein the compound of formula (6) or a pharmaceutically acceptable salt thereof is administered orally at a daily dose of 0.3 to 3 mg.

A further embodiment of the invention is the compound of formula (6) or a pharmaceutically acceptable salt thereof for use in the treatment of cognitive impairment in a mammal in need of such treatment, wherein the orally administered daily dose is not significantly reducing blood pressure.

A further embodiment of the invention is the compound of formula (6) or a pharmaceutically acceptable salt thereof for use in the treatment of cognitive impairment in a mammal in need of such treatment, wherein the compound of formula (6) administered orally at a daily dose of 0.2 to 6 mg or 0.3 to 3 mg and this dose is not significantly reducing blood pressure.

As used herein, the term “stimulator” of soluble Guanylyl Cyclase (sGC) relates to an active compound that interacts with the native, heme containing sGC, to activate the latter to catalyze the formation of cGMP (Stasch and Hobbs 2009).

As used herein, the term “stimulation” is to be understood as increasing the measured production of cGMP by at least 5% as compared to a control, e.g., a non-treated control, preferably by at least 10%, more preferably by at least 15%, even more preferably by at least 20%, even more preferably by at least 25%, even more preferably by at least 30% or by at least 40% or by at least 50%. Suitable controls are evident for the skilled person when considering the teaching of the present disclosure. Suitable assays to determine said stimulation are readily available to the skilled person from the pertinent literature. In one embodiment of the invention, assay A-3 referred to herein below is being used to determine said stimulation.

The term “cognitive impairment” refers to any decline in one or more of memory functions, decision making, executive functions, language skills, visuospatial skills, or attentional control.

The term “treating” or “treatment” as used in the present invention refers to alleviating or abrogating the cause and/or effects or symptoms or clinical manifestations of the disorder or disease. More specifically, as used herein, the terms “treating” or “treatment” refer to the reduction or amelioration or slowing down of the progression, severity and/or duration of cognitive impairment. In some embodiments, the terms “treating” or “treatment” refer to the reduction, amelioration or slowing down of the progression, the severity and/or the duration of one or more physical symptoms or clinical manifestations (preferably, one or more measurable physical symptoms or clinical manifestations) of the condition, as a result of the administration of one or more therapies (e.g., an sGC activator or an sGC stimulator selected from one of the groups specified above or a pharmaceutically acceptable salt thereof, either alone or in combination therapy). In some embodiments, “treating” or “treatment” may result in total or partial reversal of the disease (i.e., as determined by normalization of the clinical parameters, findings or manifestations associated with the disease). In other embodiments, “treating” or “treatment” may result in slowing down or halting the progression of cognitive impairment. For example, this can include the following: arresting or delaying the decline, or providing improvement in: a) memory (short-term and/or long term), b) decision making, c) executive functions (e.g., reasoning, problem-solving, planning), d) language skills (e.g. naming, fluency, expressive speech, and comprehension), e) visuospatial skills, and f) attentional control.

In some embodiments, the terms “treating” or “treatment” refer to delaying the onset of cognitive impairment in a patient in need thereof. In some embodiments, the terms “treating” or “treatment” refer to delaying the onset of a physical symptom or set of physical symptoms or clinical manifestations or findings associated with cognitive impairment.

Treatment can involve administering a compound, combination, composition or medicament described herein to a patient diagnosed with cognitive impairment and may involve administering the compound to a patient who does not have active symptoms. Conversely, treatment may involve administering the compound, combination, composition or medicament to a patient at risk of developing cognitive impairment, or to a patient reporting one or more of the physiological symptoms of the disease, even though a diagnosis of this disease may not have been made.

The compounds described in the present invention are therefore in particular suitable for treating cognitive impairment such as mild cognitive impairment, dementia, such as vascular dementia, and Alzheimer dementia by e.g. improving perception, capacity for concentration, capacity for learning or memory performance after cognitive disturbances.

Furthermore, the compounds according to the invention are suitable for controlling cerebral perfusion and are effective agents for combating migraines. Therefore the compounds are also suitable for treating cognitive impairment associated with cerebral infarctions (apoplexia cerebri) such as stroke, cerebral ischemia, ischemic stroke and head injury.

The compounds described in the present invention are therefore also suitable for improving cognitive impairment associated with head injury, stroke, post-stroke dementia, post-traumatic head injury, general disturbances of concentration, disturbances of concentration in children with learning and memory problems, Lewy body dementia, dementia with frontal lobe degeneration including Pick's syndrome, Parkinson's disease, progressive nuclear palsy, dementia with corticobasal degeneration, amyotrophic lateral sclerosis (ALS), Huntington's disease, demyelination, multiple sclerosis, thalamic degeneration, Creutzfeldt-Jakob dementia, HIV-dementia, schizophrenia with dementia or Korsakoff psychosis.

One embodiment of the invention is at least one sGC activator or a pharmaceutically acceptable salt thereof for use in the treatment of cognitive impairment in a mammal in need of such treatment.

A further embodiment of the invention is at least one sGC activator or a pharmaceutically acceptable salt thereof for use in the treatment of cognitive impairment in a mammal in need of such treatment, wherein the cognitive impairment is selected from the group consisting of mild cognitive impairment, dementia, vascular dementia, Alzheimer dementia and cognitive impairment associated with cerebral infarctions, cerebral ischemia and ischemic stroke.

A further embodiment of the invention is at least one sGC activator or a pharmaceutically acceptable salt thereof or a pharmaceutically acceptable salt thereof for use in the treatment of cognitive impairment in a mammal in need of such treatment, wherein cognitive impairment is vascular dementia.

A further embodiment of the invention is a compound of any of formulae (1) to (4) or a pharmaceutically acceptable salt thereof for use in the treatment of cognitive impairment in a mammal in need of such treatment.

A further embodiment of the invention is a compound of any of formulae (1) to (4) or a pharmaceutically acceptable salt thereof for use in the treatment of cognitive impairment in a mammal in need of such treatment, wherein cognitive impairment is selected from the group consisting of mild cognitive impairment, dementia, vascular dementia, Alzheimer dementia and cognitive impairment associated with cerebral infarctions, cerebral ischemia and ischemic stroke.

A further embodiment of the invention is a compound of any of formulae (1) to (4) or a pharmaceutically acceptable salt thereof for use in the treatment of cognitive impairment in a mammal in need of such treatment, wherein cognitive impairment is vascular dementia.

A further embodiment of the invention is a compound of any of formula (1) or a pharmaceutically acceptable salt thereof for use in the treatment of cognitive impairment in a mammal in need of such treatment.

A further embodiment of the invention is the compound of formula (1) or a pharmaceutically acceptable salt thereof for use in the treatment of cognitive impairment in a mammal in need of such treatment, wherein cognitive impairment is selected from the group consisting of mild cognitive impairment, dementia, vascular dementia, Alzheimer dementia and cognitive impairment associated with cerebral infarctions, cerebral ischemia and ischemic stroke.

A further embodiment of the invention is the compound of formula (1) or a pharmaceutically acceptable salt thereof for use in the treatment of cognitive impairment in a mammal in need of such treatment, wherein cognitive impairment is vascular dementia.

A further embodiment of the invention is a compound of any of formulae (1) to (6) or a pharmaceutically acceptable salt thereof for use in the treatment of cognitive impairment in a mammal in need of such treatment.

A further embodiment of the invention is a compound of any of formulae (1) to (6) or a pharmaceutically acceptable salt thereof for use in the treatment of cognitive impairment in a mammal in need of such treatment, wherein cognitive impairment is selected from the group consisting of mild cognitive impairment, dementia, vascular dementia, Alzheimer dementia and cognitive impairment associated with cerebral infarctions, cerebral ischemia and ischemic stroke.

A further embodiment of the invention is a compound of any of formulae (1) to (6) or a pharmaceutically acceptable salt thereof for use in the treatment of cognitive impairment in a mammal in need of such treatment, wherein cognitive impairment is vascular dementia.

A further embodiment of the invention is at least one sGC stimulator selected from one of the groups specified above for use in the treatment of cognitive impairment associated with cerebral infarctions, stroke, cerebral ischemia, ischemic stroke, head injury, post-stroke dementia, post-traumatic head injury, general disturbances of concentration, disturbances of concentration in children with learning, memory problems, Lewy body dementia, dementia with frontal lobe degeneration including Pick's syndrome, Parkinson's disease, progressive nuclear palsy, dementia with corticobasal degeneration, amyotrophic lateral sclerosis, Huntington's disease, demyelination, multiple sclerosis, thalamic degeneration, Creutzfeldt-Jakob dementia, HIV-dementia, schizophrenia with dementia or Korsakoff psychosis.

A further embodiment of the invention is at least one sGC stimulator selected from one of the groups specified above or a pharmaceutically acceptable salt thereof for use in the treatment of cognitive impairment in a mammal in need of such treatment.

A further embodiment of the invention is at least one sGC stimulator selected from one of the groups specified above or a pharmaceutically acceptable salt thereof for use in the treatment of cognitive impairment in a mammal in need of such treatment, wherein cognitive impairment is selected from the group consisting of mild cognitive impairment, dementia, vascular dementia, Alzheimer dementia and cognitive impairment associated with cerebral infarctions, cerebral ischemia and ischemic stroke.

A further embodiment of the invention is at least one sGC stimulator selected from one of the groups specified above or a pharmaceutically acceptable salt thereof for use in the treatment of cognitive impairment in a mammal in need of such treatment, wherein cognitive impairment is vascular dementia.

A further embodiment of the invention is a compound of formula (5) or (6) or a pharmaceutically acceptable salt thereof for use in the treatment of cognitive impairment in a mammal in need of such treatment.

A further embodiment of the invention is a compound of formula (5) or (6) or a pharmaceutically acceptable salt thereof for use in the treatment of cognitive impairment in a mammal in need of such treatment, wherein cognitive impairment is selected from the group consisting of mild cognitive impairment, dementia, vascular dementia, Alzheimer dementia and cognitive impairment associated with cerebral infarctions, cerebral ischemia and ischemic stroke.

A further embodiment of the invention is a compound of formula (5) or (6) or a pharmaceutically acceptable salt thereof for use in the treatment of cognitive impairment in a mammal in need of such treatment, wherein cognitive impairment is vascular dementia.

A further embodiment of the invention is a compound of formula (5) or a pharmaceutically acceptable salt thereof for use in the treatment of cognitive impairment in a mammal in need of such treatment.

A further embodiment of the invention is a compound of formula (5) or a pharmaceutically acceptable salt thereof for use in the treatment of cognitive impairment in a mammal in need of such treatment, wherein cognitive impairment is selected from the group consisting of mild cognitive impairment, dementia, vascular dementia, Alzheimer dementia and cognitive impairment associated with cerebral infarctions, cerebral ischemia and ischemic stroke.

A further embodiment of the invention is a compound of formula (5) or a pharmaceutically acceptable salt thereof for use in the treatment of cognitive impairment in a mammal in need of such treatment, wherein cognitive impairment is vascular dementia.

A further embodiment of the invention is a compound of formula (6) or a pharmaceutically acceptable salt thereof for use in the treatment of cognitive impairment in a mammal in need of such treatment.

A further embodiment of the invention is a compound of formula (6) or a pharmaceutically acceptable salt thereof for use in the treatment of cognitive impairment in a mammal in need of such treatment, wherein cognitive impairment is selected from the group consisting of mild cognitive impairment, dementia, vascular dementia, Alzheimer dementia and cognitive impairment associated with cerebral infarctions, cerebral ischemia and ischemic stroke.

A further embodiment of the invention is a compound of formula (6) or a pharmaceutically acceptable salt thereof for use in the treatment of cognitive impairment in a mammal in need of such treatment, wherein cognitive impairment is vascular dementia.

The compounds according to the invention can be used alone or in combination with other active substances if necessary. The present invention further relates to medicinal products containing at least one of the compounds according to the invention and one or more further active substances, in particular for the treatment of the aforementioned diseases. Active substances that are particularly suitable for combinations are for example and preferably:
organic nitrates and NO-donors, for example sodium nitroprusside, nitroglycerin, isosorbide mononitrate, isosorbide dinitrate, molsidomine or SIN-1, and inhalational NO;
compounds that inhibit the degradation of cyclic guanosine monophosphate (cGMP) and/or cyclic adenosine monophosphate (cAMP), for example inhibitors of phosphodiesterases (PDE) 1, 2, 3, 4 and/or 5, in particular PDE 4 inhibitors such as roflumilast or revamilast and PDE 5 inhibitors such as sildenafil, vardenafil, tadalafil, udenafil, dasantafil, avanafil, mirodenafil or lodenafil;
antiinflammatory and/or immunosuppressive compounds for example and preferably systemically or inhalatively administered corticosteroides, flutiform, pirfenidone, acetylcysteine, azathioprine or BIBF-1120;
sGC Stimulators selected from
methyl{4,6-diamino-2-[5-fluoro-1-(2-fluorobenzyl)-1H-pyrazolo[3,4-b]pyridine-3-yl]pyrimidin-5-yl}carbamate (Vericiguat) compound of formula (5)
ent-N-[(2S)-amino-2-methylbutyl]-8-[(2,6-difluorobenzyl)oxy]-2,6-dimethylimidazo[1,2-a]pyridine-3-carboxamide (enantiomer A) compound of formula (6).
ent-N-(2-amino-2-methylbutyl)-8-[(2,6-difluorobenzyl)oxy]-2,6-dimethylimidazo[1,2-a]pyridine-3-carboxamide (enantiomer B)
1,1,1,3,3,3-Hexafluoro-2-[({5-fluoro-2-[1-(2-fluorobenzyl)-5-(1,2-oxazol-3-yl)-1H-pyrazol-3-yl]-4-pyrimidinyl}amino)methyl]-2-propanol (Praliciguat)
sGC Activators selected from
3-(4-chloro-3-{[(2S,3R)-2-(4-chlorophenyl)-4,4,4-trifluoro-3-methylbutanoyl]amino}phenyl)-3-cyclopropylpropanoic acid (compound of formula (1))
4-({(4-carboxybutyl)[2-(5-fluoro-2-{[4′-(trifluoromethyl)[1,1′-biphenyl]-4-yl]methoxy}phenyl)ethyl]amino}methyl)benzoic acid (compound of formula (2))
5-{[2-(4-carboxyphenyl)ethyl][2-(2-{[3-chloro-4′-(trifluoromethyl)biphenyl-4-yl]methoxy}phenyl)ethyl]amino}-5,6,7,8-tetrahydroquinoline-2-carboxylic acid (compound of formula (4)) and
5-{(4-carboxybutyl)[2-(2-{[3-chloro-4′-(trifluoromethyl)biphenyl-4-yl]methoxy}phenyl)ethyl]amino}-5,6,7,8-tetrahydroquinoline-2-carboxylic acid (compound of formula (3))
sGC modulator IW6463
Acetylcholinesterase inhibitors, such as donepezil, rivastigmine and galantamine
NMDA receptor antagonists, such as memantine
Ergot derivatives, such as hydergine and nicergoline
compounds for lowering blood pressure, for example and preferably from the group of calcium antagonists, for example and preferably nifedipine, amlodipine, nimodipine, verapamil or diltiazem, angiotensin AII antagonists, for example and preferably losartan, candesartan, valsartan, telmisartan or embursatan, ACE inhibitors, for example and preferably enalapril, captopril, lisinopril, ramipril, delapril, fosinopril, quinopril, perindopril or trandopril, endothelin antagonists, renin inhibitors, for example and preferably aliskiren, SPP-600 or SPP-800, alpha-blockers, for example and preferably prazosin, beta-blockers, for example and preferably propranolol, atenolol, timolol, pindolol, alprenolol, oxprenolol, penbutolol, bupranolol, metipranolol, nadolol, mepindolol, carazolol, sotalol, metoprolol, betaxolol, celiprolol, bisoprolol, carteolol, esmolol, labetalol, carvedilol, adaprolol, landiolol, nebivolol, epanolol or bucindolol, mineralocorticoid receptor antagonists, for example and preferably spironolactone, eplerenone or finerenone, and diuretics, for example and preferably furosemide, bumetanide, Torsemide, bendroflumethiazide, chlorthiazide, hydrochlorthiazide, hydroflumethiazide, methyclothiazide, polythiazide, trichlormethiazide, chlorthalidone, indapamide, metolazone, quinethazone, acetazolamide, dichlorphenamide, methazolamide, glycerol, isosorbide, mannitol, amiloride or triamterene.
antithrombotic compounds, for example and preferably from the group of platelet aggregation inhibitors, anticoagulants or profibrinolytic substances; antithrombotic compounds are for example and preferably aspirin, clopidogrel, ticlopidine, dipyridamole, ximelagatran, melagatran, dabigatran, bivalirudin, Clexane, tirofiban, abciximab, rivaroxaban, apixaban, fidexaban, razaxaban, fondaparinux, idraparinux, heparin or vitamin K antagonist.
compounds that alter fat metabolism, for example and preferably from the group of thyroid receptor agonists, cholesterol synthesis inhibitors such as for example and preferably HMG-CoA-reductase or squalene synthesis inhibitors, ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbers, bile acid reabsorption inhibitors and lipoprotein(a) antagonists; compounds that alter fat metabolism are for example and preferably torcetrapib, (CP-5294/4), JJT-705, CETP-vaccine (Avant), lovastatin, simvastatin, pravastatin, fluvastatin, atorvastatin, rosuvastatin, pitavastatin, avasimibe, melinamide, pactimibe, eflucimibe, SMP-797, implitapide, BMS-201038, R-103757, JTT-130, pioglitazone, rosiglitazone, ezetimibe, tiqueside, pamaqueside, orlistat, cholestyramine, colestipol, colesolvam, CholestaGel, colestimide, gemcabene calcium (CI-1027) or nicotinic acid.
antioxidants and free-radical scavengers;
antidiabetic compounds, by way of example and with preference from the group of the insulins and insulin derivatives, sulphonylureas, biguanides, meglitinide derivatives, glucosidase inhibitors, PPAR-gamma agonists, GLP 1 receptor agonists, glucagon antagonists, insulin sensitizers, CCK1 receptor agonists, leptin receptor agonists, potassium channel antagonists and the inhibitors of hepatic enzymes that are involved in the stimulation of gluconeogenesis and/or glycogenolysis;
Vitamin E
Omega-3 fatty acids
Ginkgo, in particular Ginkgo biloba extracts The combinations described in the present invention are therefore effective for controlling diseases in the central nervous system with unexpected beneficial properties compared to the state of the art.

A further embodiment of the invention is a combination of at least one sGC activator or a pharmaceutically acceptable salt thereof or sGC stimulator selected from one of the groups specified above or a pharmaceutically acceptable salt thereof and at least one compound selected from the group consisting of organic nitrates and NO-donors, inhibitors of phosphodiesterases (PDE) 1, 2, 3, 4 or 5, anti-inflammatory compounds, immunosuppressive compounds, acetylcholinesterase inhibitors, NMDA receptor antagonists, compounds suitable for lowering blood pressure, antithrombotic compounds, compounds suitable for altering fat metabolism and antidiabetic compounds for use in the treatment of cognitive impairment in a mammal in need of such treatment.

A further embodiment of the invention is a combination of at least one sGC activator or a pharmaceutically acceptable salt thereof and at least one compound selected from the group consisting of organic nitrates and NO-donors, inhibitors of phosphodiesterases (PDE) 1, 2, 3, 4 or 5, anti-inflammatory compounds, immunosuppressive compounds, acetylcholinesterase inhibitors, NMDA receptor antagonists, compounds suitable for lowering blood pressure, antithrombotic compounds, compounds suitable for altering fat metabolism and antidiabetic compounds for use in the treatment of cognitive impairment in a mammal in need of such treatment.

A further embodiment of the invention is a combination of compounds of formula (1) to (6) or a pharmaceutically acceptable salt thereof and at least one compound selected from the group consisting of organic nitrates and NO-donors, inhibitors of phosphodiesterases (PDE) 1, 2, 3, 4 or 5, anti-inflammatory compounds, immunosuppressive compounds, acetylcholinesterase inhibitors, NMDA receptor antagonists, compounds suitable for lowering blood pressure, antithrombotic compounds, compounds suitable for altering fat metabolism and antidiabetic compounds for use in the treatment of cognitive impairment in a mammal in need of such treatment.

A further embodiment of the invention is a combination comprising one or more compounds of formulae (1) to (4) or a pharmaceutically acceptable salt thereof and at least one compound selected from the group consisting of organic nitrates and NO-donors, inhibitors of phosphodiesterases (PDE) 1, 2, 3, 4 or 5, anti-inflammatory compounds, immunosuppressive compounds, acetylcholinesterase inhibitors, NMDA receptor antagonists, compounds suitable for lowering blood pressure, antithrombotic compounds, compounds suitable for altering fat metabolism and antidiabetic compounds for use in the treatment of cognitive impairment in a mammal in need of such treatment.

A further embodiment of the invention is a combination comprising the compound of formula (1) or a pharmaceutically acceptable salt thereof and at least one compound selected from the group consisting of organic nitrates and NO-donors, inhibitors of phosphodiesterases (PDE) 1, 2, 3, 4 or 5, anti-inflammatory compounds, immunosuppressive compounds, acetylcholinesterase inhibitors, NMDA receptor antagonists, compounds suitable for lowering blood pressure, antithrombotic compounds, compounds suitable for altering fat metabolism and antidiabetic compounds for use in the treatment of cognitive impairment in a mammal in need of such treatment.

A further embodiment of the invention is a combination comprising one or more compounds of formulae (5) and (6) or a pharmaceutically acceptable salt thereof and at least one compound selected from the group consisting of organic nitrates and NO-donors, inhibitors of phosphodiesterases (PDE) 1, 2, 3, 4 or 5, anti-inflammatory compounds, immunosuppressive compounds, acetylcholinesterase inhibitors, NMDA receptor antagonists, compounds suitable for lowering blood pressure, antithrombotic compounds, compounds suitable for altering fat metabolism and antidiabetic compounds for use in the treatment of cognitive impairment in a mammal in need of such treatment.

A further embodiment of the invention is a combination comprising the compound of formula (5) or a pharmaceutically acceptable salt thereof and at least one compound selected from the group consisting of organic nitrates and NO-donors, inhibitors of phosphodiesterases (PDE) 1, 2, 3, 4 or 5, anti-inflammatory compounds, immunosuppressive compounds, acetylcholinesterase inhibitors, NMDA receptor antagonists, compounds suitable for lowering blood pressure, antithrombotic compounds, compounds suitable for altering fat metabolism and antidiabetic compounds for use in the treatment of cognitive impairment in a mammal in need of such treatment.

A further embodiment of the invention is a combination comprising the compound of formula (6) or a pharmaceutically acceptable salt thereof and at least one compound selected from the group consisting of organic nitrates and NO-donors, inhibitors of phosphodiesterases (PDE) 1, 2, 3, 4 or 5, anti-inflammatory compounds, immunosuppressive compounds, acetylcholinesterase inhibitors, NMDA receptor antagonists, compounds suitable for lowering blood pressure, antithrombotic compounds, compounds suitable for altering fat metabolism and antidiabetic compounds for use in the treatment of cognitive impairment in a mammal in need of such treatment.

A further embodiment of the invention is a combination of at least one sGC activator or a pharmaceutically acceptable salt thereof or sGC stimulator selected from one of the groups specified above or a pharmaceutically acceptable salt thereof and at least one compound selected from the group consisting of donepezil, rivastigmine, galantamine and memantine for use in the treatment of cognitive impairment in a mammal in need of such treatment.

A further embodiment of the invention is a combination of at least one sGC activator or a pharmaceutically acceptable salt thereof and at least one compound selected from the group consisting of donepezil, rivastigmine, galantamine and memantine for use in the treatment of cognitive impairment in a mammal in need of such treatment.

A further embodiment of the invention is a combination of compounds of formulae (1) to (6) or a pharmaceutically acceptable salt thereof and at least one compound selected from the group consisting donepezil, rivastigmine, galantamine and memantine for use in the treatment of cognitive impairment in a mammal in need of such treatment.

A further embodiment of the invention is a combination comprising one or more compounds of formulae (1) to (4) or a pharmaceutically acceptable salt thereof and at least one compound selected from the group consisting of donepezil, rivastigmine, galantamine and memantine for use in the treatment of cognitive impairment in a mammal in need of such treatment.

A further embodiment of the invention is a combination comprising the compound of formula (1) or a pharmaceutically acceptable salt thereof and at least one compound selected from the group consisting of donepezil, rivastigmine, galantamine and memantine for use in the treatment of cognitive impairment in a mammal in need of such treatment.

A further embodiment of the invention is a combination comprising one or more compounds of formulae (5) and (6) or a pharmaceutically acceptable salt thereof and at least one compound selected from the group consisting of donepezil, rivastigmine, galantamine and memantine for use in the treatment of cognitive impairment in a mammal in need of such treatment.

A further embodiment of the invention is a combination comprising the compound of formula (5) or a pharmaceutically acceptable salt thereof and at least one compound selected from the group consisting of donepezil, rivastigmine, galantamine and memantine for use in the treatment of cognitive impairment in a mammal in need of such treatment.

A further embodiment of the invention is a combination comprising the compound of formula (6) or a pharmaceutically acceptable salt thereof and at least one compound selected from the group consisting of donepezil, rivastigmine, galantamine and memantine for use in the treatment of cognitive impairment in a mammal in need of such treatment.

A further embodiment of the invention is a medicament comprising one or more sGC activator or a pharmaceutically acceptable salt thereof or sGC stimulator selected from one of the groups specified above or a pharmaceutically acceptable salt thereof in a dose described above or a combination as described above for use in the treatment of cognitive impairment in a mammal in need of such treatment, wherein cognitive impairment is selected from the group consisting of mild cognitive impairment, dementia, vascular dementia, Alzheimer dementia and cognitive impairment associated with cerebral infarctions, cerebral ischemia and ischemic stroke.

A further embodiment of the invention is a medicament comprising one or more sGC activator or a pharmaceutically acceptable salt thereof in a dose described above or a combination as described above for use in the treatment of cognitive impairment in a mammal in need of such treatment, wherein cognitive impairment is selected from the group consisting of mild cognitive impairment, dementia, vascular dementia, Alzheimer dementia and cognitive impairment associated with cerebral infarctions, cerebral ischemia and ischemic stroke.

A further embodiment of the invention is a medicament comprising one or more compounds of formulae (1) to (6) or a pharmaceutically acceptable salt thereof in a dose described above or a combination as described above for use in the treatment of cognitive impairment in a mammal in need of such treatment, wherein cognitive impairment is selected from the group consisting of mild cognitive impairment, dementia, vascular dementia, Alzheimer dementia and cognitive impairment associated with cerebral infarctions, cerebral ischemia and ischemic stroke.

A further embodiment of the invention is a medicament comprising one or more compounds of formulae (1) to (4) or a pharmaceutically acceptable salt thereof in a dose described above or a combination as described above for use in the treatment of cognitive impairment in a mammal in need of such treatment, wherein cognitive impairment is selected from the group consisting of mild cognitive impairment, dementia, vascular dementia, Alzheimer dementia and cognitive impairment associated with cerebral infarctions, cerebral ischemia and ischemic stroke.

A further embodiment of the invention is a medicament comprising the compound of formula (1) or a pharmaceutically acceptable salt thereof in a dose described above or a combination as described above for use in the treatment of cognitive impairment in a mammal in need of such treatment, wherein cognitive impairment is selected from the group consisting of mild cognitive impairment, dementia, vascular dementia, Alzheimer dementia and cognitive impairment associated with cerebral infarctions, cerebral ischemia and ischemic stroke.

A further embodiment of the invention is a medicament comprising one or more compounds of formulae (5) and (6) or a pharmaceutically acceptable salt thereof in a dose described above or a combination as described above for use in the treatment of cognitive impairment in a mammal in need of such treatment, wherein cognitive impairment is selected from the group consisting of mild cognitive impairment, dementia, vascular dementia, Alzheimer dementia and cognitive impairment associated with cerebral infarctions, cerebral ischemia and ischemic stroke.

A further embodiment of the invention is a medicament comprising the compound of formula (5) or a pharmaceutically acceptable salt thereof in a dose described above or a combination as described above for use in the treatment of cognitive impairment in a mammal in need of such treatment, wherein cognitive impairment is selected from the group consisting of mild cognitive impairment, dementia, vascular dementia, Alzheimer dementia and cognitive impairment associated with cerebral infarctions, cerebral ischemia and ischemic stroke.

A further embodiment of the invention is a medicament comprising the compound of formula (6) or a pharmaceutically acceptable salt thereof in a dose described above or a combination as described above for use in the treatment of cognitive impairment in a mammal in need of such treatment, wherein cognitive impairment is selected from the group consisting of mild cognitive impairment, dementia, vascular dementia, Alzheimer dementia and cognitive impairment associated with cerebral infarctions, cerebral ischemia and ischemic stroke.

### FIGURES

FIG. 1: Effects of either 0.03 mg/kg, 0.1 mg/kg, 0.3 mg/kg, 1.0 mg/kg, 3.0 mg/kg doses of the compound of formula (5) (sGC stimulator), or 1.0 mg/kg donepezil or vehicle, injected 30 min before T1, on the discrimination index (d2) in an object location task using a 24 h interval (means+SEM).

When compared with chance level (i.e. zero; d2=0), the compound of formula (5) (sGC stimulator) injected 30 min before T1, improved memory performance at the doses of 0.1, 0.3 and 1.0 mg/kg. One-way ANOVA and subsequent post-hoc LSD t-tests revealed significant higher memory performance at 0.3 and 1.0 mg/kg of the compound of formula (5) (sGC stimulator) when compared to the vehicle condition. Reference compound donepezil was also injected 30 min before T1 and improved memory at 1.0 mg/kg, as indicated by both one-sample t-tests and one-way ANOVA and subsequent post-hoc LSD t-tests (comparison with vehicle). A difference from zero is depicted with hashes (One sample t-tests, #: P<0.05; ##: P<0.01; ###: P<0.001) and a difference from the vehicle condition is depicted with asterisks (One-way ANOVA, LSD t-tests, *: P<0.05; **: P<0.01; ***: P<0.001).

FIG. 2: Effects of either 0.01 mg/kg, 0.03 mg/kg, 0.1 mg/kg, 0.3 mg/kg, 1.0 mg/kg, 3.0 mg/kg doses of the compound of formula (6) (sGC stimulator), or 1.0 mg/kg donepezil or vehicle, injected 30 min before T1, on the discrimination index (d2) in an object location task using a 24 h interval (means+SEM).

When compared to the vehicle condition, the compound of formula (6) (sGC stimulator), injected 30 min before T1, improved memory performance at the doses of 0.03, 0.1, 0.3 and 1.0 mg/kg. Reference compound donepezil was also injected 30 min before T1 and improved memory at 1.0 mg/kg, as indicated by both one-sample t-tests and the one-way ANOVA. A difference from zero is depicted with hashes (One sample t-tests, ##: P<0.01; ###: P<0.001). A difference from the vehicle condition is depicted with asterisks (One-way ANOVA, LSD t-tests, **: P<0.01; ***: P<0.001).

FIG. 3: Effects of either 0.01 mg/kg, 0.03 mg/kg, 0.1 mg/kg, 0.3 mg/kg, 1.0 mg/kg, 3.0 mg/kg doses of the compound of formula (1), or 1.0 mg/kg donepezil or vehicle, injected 30 min before T1, on the discrimination index (d2) in an object location task using a 24 h interval (means+SEM).

When compared with chance level (i.e. zero; d2=0), the compound of formula (1) injected 30 min before T1, improved memory performance at the doses of 0.03, 0.1, 0.3 and 1.0 mg/kg. One-way ANOVA and subsequent post-hoc LSD t-tests revealed significant higher memory performance at 0.1, 0.3 and 1.0 mg/kg of the compound of formula (1) when compared to the vehicle condition. Reference compound donepezil was also injected 30 min before T1 and improved memory at 1.0 mg/kg, as indicated by both one-sample t-tests and one-way ANOVA and subsequent post-hoc LSD t-tests (comparison with vehicle). A difference from zero is depicted with hashes (One sample t-tests, #: P<0.05; ###: P<0.001). A difference from the vehicle condition is depicted with asterisks (One-way ANOVA, LSD t-tests, *: P<0.05; **: P<0.01; ***: P<0.001).

FIG. 4: Effects of either 0.03 mg/kg, 0.1 mg/kg, 0.3 mg/kg, 1.0 mg/kg, 3.0 mg/kg doses of the compound of formula (2), or 1.0 mg/kg donepezil or vehicle, injected 30 min before T1, on the discrimination index (d2) in an object location task using a 24 h interval (means+SEM).

When compared with chance level (i.e. zero; d2=0), the compound of formula (2) injected 30 min before T1, improved memory performance at the doses of 0.1, 0.3 and 1.0 mg/kg. One-way ANOVA and subsequent post-hoc LSD t-tests revealed significant higher memory performance at 0.3 and 1.0 mg/kg of the compound of formula (2) when compared to the vehicle condition. Reference compound donepezil was also injected 30 min before T1 and improved memory at 1.0 mg/kg, as indicated by both one-sample t-tests and one-way ANOVA and subsequent post-hoc LSD t-tests (comparison with vehicle). A difference from zero is depicted with hashes (One sample t-tests, ##: P<0.01; ###: P<0.001) and a difference from the vehicle condition is depicted with asterisks (One-way ANOVA, LSD t-tests, *: P<0.05; **: P<0.01; ***: P<0.001).

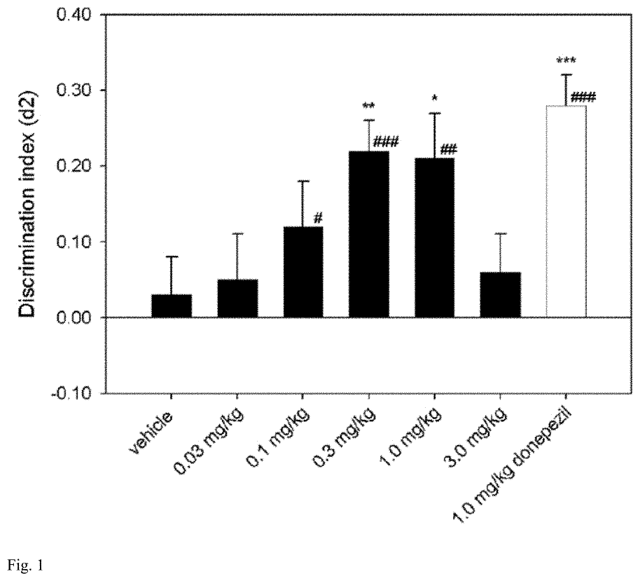

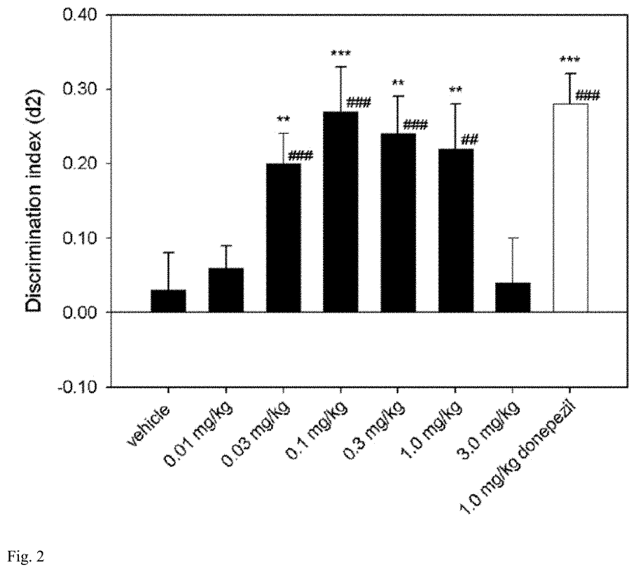

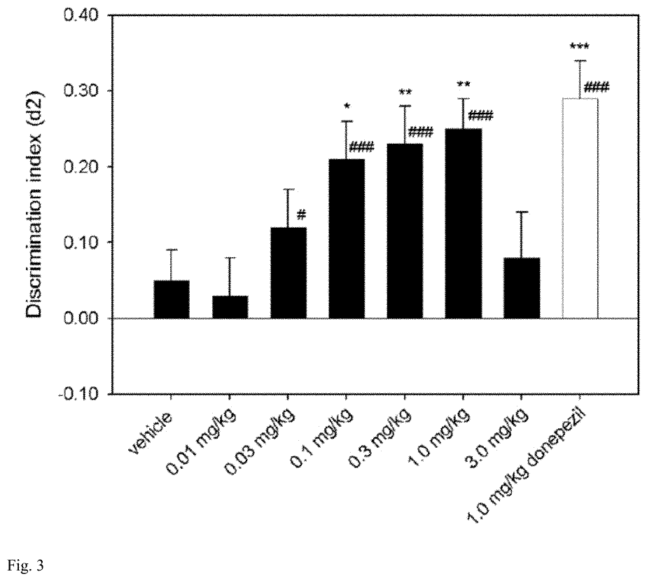

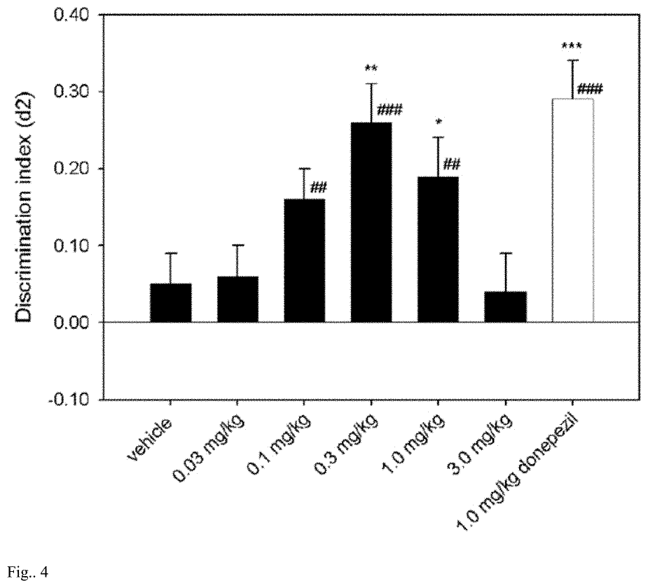

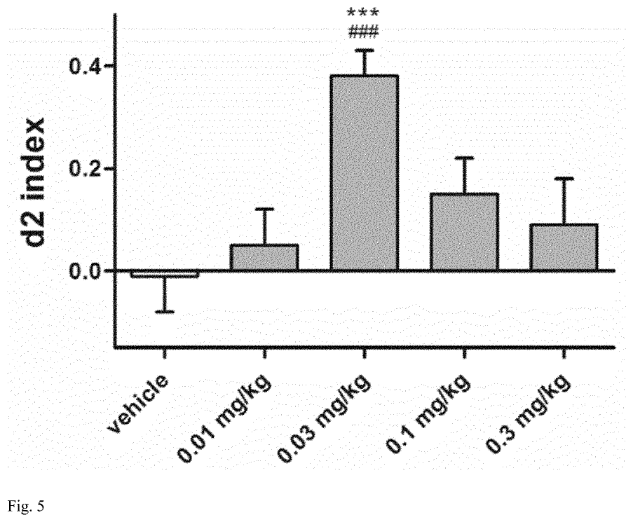

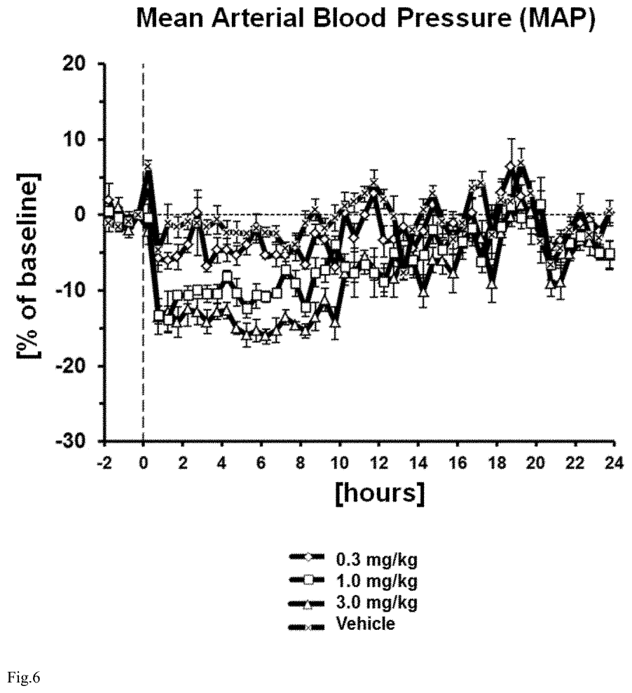

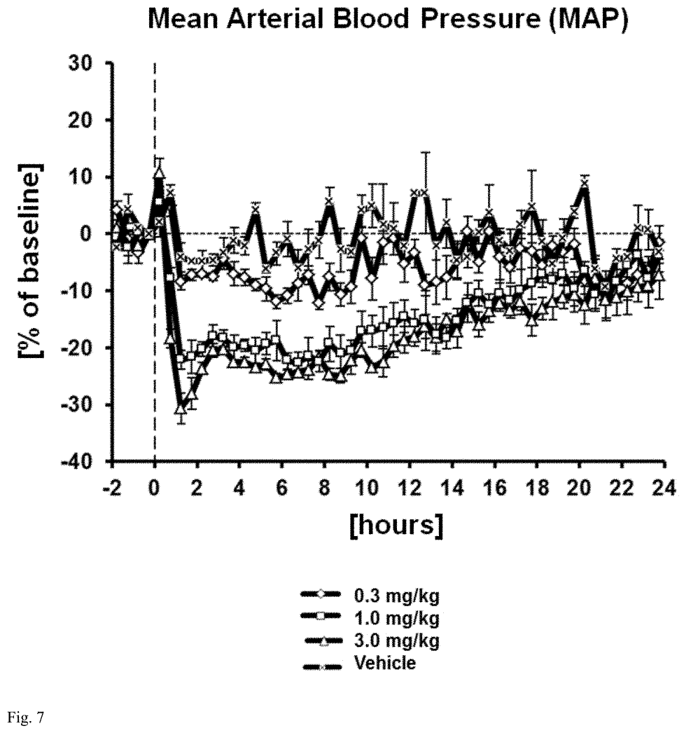

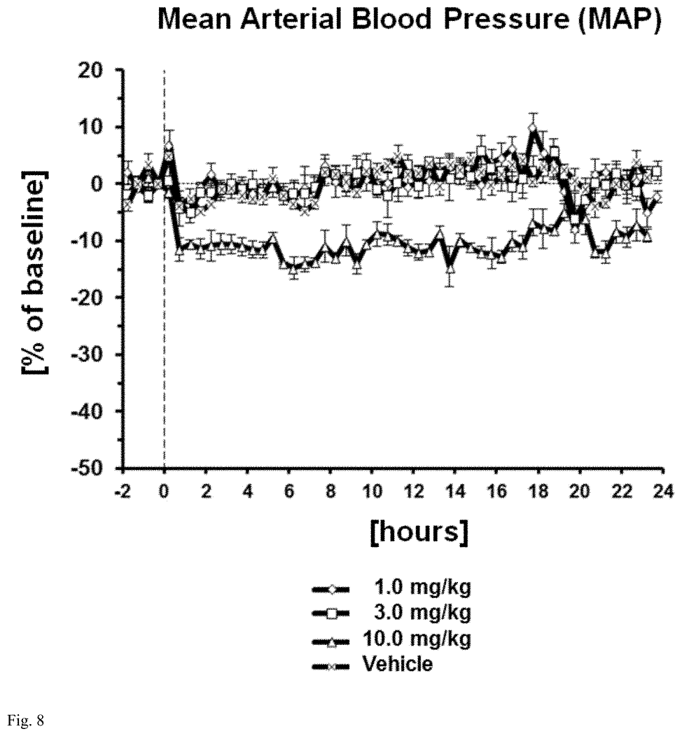

FIG. 5: (Comparative example): Effects of the sGC stimulator riociguat, as exemplified in FIG. 1 of WO 2017/108441 A1.

FIG. 6: Effects of 0.3 mg/kg, 1.0 mg/kg, or 3.0 mg/kg doses of the compound of formula (5) (sGC stimulator), injected at T=0 hours on the mean arterial blood pressure over 24 hours (means+SEM).

When compared to injection of vehicle, the compound of formula (5) caused a dose-dependent effect on mean arterial blood pressure (MAP). MAP was significantly lowered at a dose of 1.0 and 3.0 mg/kg. However, the compound of formula (5) caused no significant decrease in MAP in the 0.3 mg/kg dose.

These data suggest that the compound of formula (5) has the most prominent effect in the memory test—which was seen after application of 0.3 mg/kg (FIG. 1)—at a dose which does not decrease blood pressure.

FIG. 7: Effects of 0.3 mg/kg, 1.0 mg/kg, or 3.0 mg/kg doses of the compound of formula (6) (sGC stimulator), injected at T=0 hours on the mean arterial blood pressure over 24 hours (means+SEM).

When compared to injection of vehicle, the compound of formula (6) caused a dose-dependent effect on mean arterial blood pressure (MAP). MAP was significantly lowered at a dose of 1.0 and 3.0 mg/kg. However, the compound of formula (6) caused no significant decrease in MAP at a dose of 0.3 mg/kg.

These data suggest that the compound of formula (6) has the most prominent effect in the memory test—which was seen after application of 0.1 and 0.3 mg/kg (FIG. 2)—at doses which do not decrease blood pressure.

FIG. 8: Effects of 1.0 mg/kg, 3.0 mg/kg, or 10 mg/kg doses of the compound of formula (1), injected at T=0 hours on the mean arterial blood pressure over 24 hours (means+SEM).

When compared to injection of vehicle, the compound of formula (1) caused a dose-dependent effect on mean arterial blood pressure (MAP). MAP was significantly lowered at a dose of 10.0 mg/kg. However, the compound of formula (1) caused no significant decrease in MAP at a dose of 1.0 and 3.0 mg/kg.

These data suggest that the compound of formula (1) has the most prominent effects in the memory test—which was seen after application of 0.3 (FIG. 3)—at a dose which do not decrease blood pressure.

### A) ASSESSMENT OF PHYSIOLOGICAL EFFICACY

A-1) Test for Learning and Memory Improvement In Vivo

The aim of the non-clinical studies was to test the effects of the compounds of formula (5) and (6) (sGC stimulators) and the sGC activators (compounds of formula (1) and (2)) on learning and memory improvement. Therefore, the well accepted Object Location Task (OLT) in rats was used. This task allows the assessment of acquisition, consolidation and retrieval of (spatial) information into memory, and is derived from the Object Recognition Task (ORT) (e.g. Ennaceur and Delacour, 1988; Prickaerts et al., 1997).

In detail:

Animals

All experimental procedures were approved by the local ethical committee of Maastricht University for animal experiments and met governmental guidelines. Twenty-four 3-4-months-old male Wistar rats (Charles River, Sulzfeld, Germany) were used (average body weight at the beginning of the study: cohort 1 (compounds of formula (5) and (6)): 292 g; cohort 2 (compound of formula (1) and (2)): 334 g). The animals were housed individually in a standard IVC cage system on sawdust bedding in an air-conditioned room (about 20° C.). They were kept under a reversed 12/12 h light/dark cycle (lights on from 19.00 to 07.00) and had free access to food and water. Rats were housed and tested in the same room. A radio, which was playing softly, provided background noise in the room. All testing was done between 09.00 and 17.00.

Treatment

The test compounds were freshly prepared on every experimental day and were dissolved in 0.5% Tylose solution (98% of the end volume) with 2% Tween80. Donepezil was also prepared fresh on every experimental day and was dissolved in saline.

The compound of formula (5) was tested at doses of 0, 0.03, 0.1, 0.3, 1.0 and 3.0 mg/kg, the compound of formula (6) was tested at doses of 0, 0.01, 0.03, 0.1, 0.3, 1.0 and 3.0 mg/kg, the compound of formula (2) was tested at doses of 0, 0.03, 0.1, 0.3, 1.0 and 3.0 mg/kg, the compound of formula (1) was tested at doses of 0, 0.01, 0.03, 0.1, 0.3, 1.0 and 3.0 mg/kg, and donepezil was tested at the dose of 1.0 mg/kg in a time-dependent memory deficit model, i.e. a 24 h inter-trial interval. The vehicle condition was tested only once per cohort, since in both cohorts, the compounds of formulae (5) and (6) (cohort 1) and compounds of formula (1) and (2) (cohort 2) were dissolved in the same vehicle. The AChEI donepezil acted as a reference drug. The compounds of formulae (5) and (6), and the compounds of formulae (2) and (1) and donepezil were administered p.o. (injection volume 2 ml/kg), 30 min before T1 to investigate the effects on the memory acquisition process. The order of the treatments was balanced to prevent the data from being distorted by potential object- and side-preferences of the animals.

Object Location Task

The Object Location Task (OLT) was derived from the Object Recognition Test (ORT) (Ennaceur and Delacour, 1988). The OLT is a one-trial learning task which allows the assessment of spatial memory, and was performed as described elsewhere (Bruno et al., 2011, Vanmierlo et al., 2011). In the first (learning) trial a rat is put into an arena in which two identical objects are placed. After a certain delay, the rat is given a second trial. In this second trial the rat is again placed in the same arena but now one of the objects has been moved to a different position within the area. In other words, a new spatial arrangement is being used.

The apparatus consisted of a circular arena, 83 cm in diameter. The back-half of the 40 cm high arena wall was made of gray polyvinyl chloride, the front-half consisted of transparent polyvinyl chloride. The light intensity was equal in the different parts of the apparatus, as fluorescent red tubes provided a constant illumination of about 20 lux on the floor of the apparatus. In the first (learning) trial (T1), two objects were placed in a symmetrical position on a distance of about 10 cm from the wall of the left- and the right-side of the arena. In the second (test) trial (T2), one object is moved to a new location which is about 20 cm higher or lower than the original positon. Four different sets of objects were used. The different objects were: 1) a cone consisting of a gray polyvinyl chloride base (maximal diameter 18 cm) with a collar on top made of aluminum (total height 16 cm), 2) a standard 1 L brown glass bottle (diameter 10 cm, height 22 cm) filled with water, 3) a massive metal cube (10.0×5.0×7.5 cm) with two holes (diameter 1.9 cm), and 4) a solid aluminum cube with a tapering top (13.0×8.0×8.0 cm). Rats were unable to displace the objects.

A testing session consisted of two trials. The duration of each trial was 3 min. During the first trial (T1) the apparatus contained two identical objects. Rats were placed in the apparatus facing the wall at the middle of the front (transparent) segment. After the first exploration period the rat was put back in its home cage. Subsequently, after a 24 h delay interval, the rat was put in the apparatus for the second trial (T2). The total time an animal spent exploring each object during T1 and T2 was recorded manually with a personal computer.

Exploration was defined as follows: directing the nose to the object at a distance of no more than 2 cm and/or touching the object with the nose. Sitting on the object was not considered as exploratory behavior. A minimal amount of object interaction is required in order to achieve reliable object discrimination, therefore rats that explore less than 7 s in T1 and/or 9 s in T2 should be excluded from the analyses (Akkerman et al., 2012). In order to avoid the presence of olfactory cues, the objects were always thoroughly cleaned after each trial with a 70% ethanol solution. All objects as well as the locations (left or right) of the objects were used in a balanced manner to avoid potential biases due to preferences for particular locations or objects.

In several studies it was shown that Wistar rats show a good object-location memory performance when a 1 h delay is interposed between the first trial and the second trial. However, when a 24 h delay is used rats do not discriminate between the novel and the familiar object-location in the second trial, indicating that the rats do not remember the object-location that was presented in the first trial. Using a 6 h delay, the discrimination performance is in-between than of the 1 h and 24 h delays, suggesting a delay-dependent forgetting in this task.

Procedure Per Cohort of Rats

In the first two weeks, the animals were handled daily and were allowed to get accustomed to the test setup in two days, i.e. they were allowed to explore the apparatus (without any objects) twice for 5 min each day. Then the rats were adapted to the testing routine until they showed a stable discrimination performance. After this, an experiment was performed in which the compound of formula (5) (cohort 1) or compound of formula (2) (cohort 2) was tested. Following this experiment, the reference compound donepezil and subsequently the compound of formula (6) (cohort 1) or the compound of formula (1) (cohort 2) were tested. All conditions were tested in 16 animals (except the vehicle conditions, which were tested in 24 animals). The compounds of formulae (5) and (6), the compound of formula (2), and the compound of formula (1) and donepezil were injected 30 min before T1 to investigate the effects of these compounds on the memory acquisition process. A 24 h inter-trial interval between T1 and T2 was used. Initially, three doses (0.1, 0.3 and 1.0 mg/kg) of the compound of formula (5) (cohort 1) or the compound of formula (2) (cohort 2) were investigated. During the experiment it was decided to test additional doses in order to further elaborate the dose-response curve. Donepezil was tested at a dose of 1.0 mg/kg in both cohorts since previous studies in our lab have shown that this is the optimal dose for donepezil to be effective orally in rats. Of note, the experimenter was always unaware of the conditions that were being tested. During testing the rats were assigned to treatment conditions in a balanced manner, thereby ensuring that all object combinations were distributed equally over the treatment conditions.

Statistical Analysis

The basic measures were the times spent by rats in exploring an object during T1 and T2. The time spent in exploring the two symmetrically placed objects in T1 will be represented by ‘a1’ and ‘a2’. The time spent in T2 in exploring the familiar and the novel object-location will be represented by ‘a3’ and ‘b’, respectively. The following variables were calculated: e1=a1+a2, e2=a3+b, and d2=(b−a3)/e2 (see Table 1). e1 and e2 are measures of the total exploration time of both objects during T1 and T2 respectively. d2 is a relative measure of discrimination corrected for exploratory activity in the test-trial (e2). Thus, even if a treatment would affect exploratory behavior, the d2 index will be comparable between conditions. One-sample t-statistics were performed in order to assess per treatment condition whether d2 differed from zero. However, comparison of the mean d2 value with the value zero may not be the most suitable way for analyzing recognition (increased chance of making a type I error). Results were therefore also assessed using one-way ANOVA. In case of a significant difference between treatment conditions, pairwise post-hoc comparisons were performed using LSD t-tests.

TABLE 1
Measures involved in the Object Location Test (OLT)

| Exploration | Discrimination |
|---|---|
| e1 = a1 + a2 | |
| e1 = a3 + b | d2 = (b − a3)/e2 |

e1 is the measure of the time spent in exploring both symmetrically placed objects (a1 and a2) in the first trial, and e2 is the measure of the time spent in exploring both the familiar- (a3) and the new object-location (b) in the second trial; d2 corresponds to the ability to discriminate between the familiar and novel object-location during the second trial and is corrected for exploration time during that trial.

Human doses were calculated from the doses administered to rats based on the formula for dose translation from animals to humans as described by Reagan-Shaw and colleagues (Reagan-Shaw et al, 2007).

A-2) Measurement of Compound Exposure in Blood and Brain In Vitro

In addition, 30 min after treatment with placebo, the compound of formula (5), the compound of formula (6), the compound of formula (2), or the compound of formula (1), blood and brain samples were collected for measurement of compound exposure in blood and brain. For the compound of formula (5), the following doses where measured: 0.03 mg/kg; 0.3 mg/kg; 3 mg/kg. For the compound of formula (6), the following doses where measured: 0.1 mg/kg; 3 mg/kg. For the compound of formula (1), the following doses where measured: 0.3 mg/kg; 3 mg/kg. For the compound of formula (2), the following doses where measured: 0.03 mg/kg; 0.3 mg/kg, 3 mg/kg.

A-3) Stimulation of Recombinant Soluble Guanylate Cyclase (sGC) In Vitro

Investigations on the modulation of recombinant soluble guanylate cyclase (sGC) by the compounds according to the invention with and without sodium nitroprusside, and with and without the heme-dependent sGC inhibitor 1H-1,2,4-oxadiazolo[4,3a]quinoxalin-1-one (ODQ), are carried out by the method described in detail in the following reference: M. Hoenicka, E. M. Becker, H. Apeler, T. Sirichoke, H. Schroeder, R. Gerzer and J.-P. Stasch, “Purified soluble guanylyl cyclase expressed in a baculovirus/Sf9 system: Stimulation by YC-1, nitric oxide, and carbon oxide”, J. Mol. Med. 77 (1999), 14-23. The heme-free guanylate cyclase is obtained by adding Tween 20 to the sample buffer (0.5% in the final concentration).

As described in WO 2012/139888, combination of sGC activators and 2-(N,N-diethylamino)diazenolate 2-oxide (DEA/NO), an NO donor, show no synergistic effect, i.e. the effect of DEA/NO is not potentiated as is expected with an sGC modulator acting via a heme-dependent mechanism. In addition, the effect of the sGC activator according to the invention is not blocked by 1H-1,2,4-oxadiazolo[4,3a]quinoxalin-1-one (ODQ), a heme-dependent inhibitor of soluble guanylate cyclase, but is in fact increased.

Thus, this test is suitable to distinguish between the heme-dependent sGC Stimulators and the heme-independent sGC Activators.

A-4) Measurement of Arterial Blood Pressure In Vivo.

Animals

Adult normotensive Wistar rats (Wistar HSD CPB:WU) with a body weight of 250 to 350 g were used for these experimental studies. All animals were housed in individual cages at 22-24° C. ambient temperature and maintained on a 12-hour light/dark cycle with free access to standard laboratory rat chow and water ad libitum. All animal experiments were done in accordance to the current national legislation (German protection of animals act and the EU directives on the protection of animals used for scientific purposes). All performed studies were approved by the regional regulatory authority (LANUV NRW in Germany) and by the institutional animal care and use committee of Bayer AG.

Test Principle

Blood pressure is monitored in freely moving conscious rats by radiotelemetry by a telemetric system (DSI Data Science International, MN, USA). A transmitters (TAI1PA-C40) is implanted in the abdomen of the rat during deep anaesthesia. After recovery of the rats, telemetric signals are registered by a receiver plate (RA1010) and compiled by a computer-based acquisition software (Dataquest A.R.T 4.1 for Windows).

Implantation of Transmitters

After shaving the abdominal wall of the rat, a midline abdominal incision was made, and the fluid-filled sensor catheter was inserted upstream into the exposed descending aorta between the iliac bifurcation and the renal arteries. According to the DSI guidelines the tip of the telemetric catheter was located just caudal to the renal arteries and secured by tissue adhesive. The transmitter body was affixed to the inner peritoneal wall before closure of abdomen. A two-layer closure of the abdominal incision was used, with individual suturing of the peritoneum and the muscle wall followed by closure of the outer skin. Surgery was performed under aseptic conditions. For postsurgical protection against infections and pain a single dosage of an antibiotic (Oxytetracyclin® 10%, 60 mg/kg s.c., 0.06 ml/100 g body weight, Beta-Pharma GmbH & Co, Germany) and analgesic were injected (Rimadyl®, 4 mg/kg s.c., Pfizer, Germany).

Measurements

After activation of the implanted transmitters, A.R.T., an on-line data acquisition system, samples data and converts telemetric pressure signals to mm Hg. A barometric pressure reference allows for relation of absolute pressure (relative to vacuum) to ambient atmospheric pressure. Data acquisition software was predefined to sample hemodynamic data for 10-s intervals every 5 minutes. Data collection to file was started 2 hours (T=−2 hours) before drug administration (T=0 hours) and finished after completion of 24 hours (T=24 hours).

Statistics

Data are expressed as % of baseline values. The baseline value for each animal was calculated and represents the mean blood pressure of the 2 hour period (T=−2 hours to T=0 hours) before the administration of drug or vehicle. For further analysis data were grouped to provide mean for every 0.5 hours. The means of all values obtained for each individual during the period indicated were averaged for each day. Groups were compared by one-way ANOVA with Dunnet's test.

Experimental Protocol

All animals were treated with single oral doses of vehicle or the compound in different dosages (e.g. 0.3, 1.0, 3.0, or 10.0 mg/kg). Application volume was 2 ml/kg body weight. The experiment started at 7:00 a.m. (T=−2 hours), vehicle or drug administration took place at 9.00 a.m. (=0 hours) and registration period was 24 hours. For analysis, data were grouped to provide half-hourly averages.

### REFERENCES

Akkerman, S., Prickaerts, J., Steinbusch, H. W. M., Blokland, A. (2012). Object recognition testing: Statistical considerations. Behavioural Brain Research, 232; 317-322
Bruno, O., Fedele, E., Prickaerts, J., Parker, L. A., Canepa, E., Brullo, C., Cavallero, A., Gardella, E., Balbi, A., Domenicotti, C., Bollen, E., Gijselaers, H. J., Vanmierlo, T., Erb, K., Limebeer, C. L., Argellati, F., Marinari, U. M., Pronzato, M. A., Ricciarelli, R., (2011). GEBR-7b, a novel PDE4D selective inhibitor that improves memory in rodents at non-emetic doses. Br J Pharmacol, 164; 2054-2063
Celikyurt K., Utkan T., Ozer C., Gacar N., Aricioglu F. (2014). Effects of YC-1 on Learning and Memory Functions of Aged Rats. Med Sci Monit Basic Res. 20: 130-137
Ennaceur, A., Delacour, J., (1988). A new one-trial test for neurobiological studies of memory in rats. 1: Behavioral data. Behav Brain Res. 31; 47-59
Evgenov O. V., Pacher P., Schmidt P. M., Haskó G., Schmidt H. H., Stasch J. P. (2006). NO-independent stimulators and activators of soluble guanylate cyclase: discovery and therapeutic potential. Nat Rev Drug Discov. 2006; September; 5(9):755-768
Follmann, M., Ackerstaff, J., Redlich, G., Wunder, F., Lang, D., Kern, A., Fey, P., Griebenow, N., Kroh, W., Becker-Pelster, E. M., Kretschmer, A., Geiss, V., Li, V., Straub, A., Mittendorf, J., Jautelat, R., Schirok, H., Schlemmer, K. H., Lustig, K., Gerisch, M., Knorr, A., Tinel, H., Mondritzki, T., Trubel, H., Sandner, P., and Stasch, J. P. (2017). Discovery of the Soluble Guanylate Cyclase Stimulator Vericiguat (BAY 1021189) for the Treatment of Chronic Heart Failure. J Med Chem. 60(12):5146-5161
Heckman P R A, Blokland A, Bollen E P P, Prickaerts J. (2018) Phosphodiesterase inhibition and modulation of corticostriatal and hippocampal circuits: Clinicalb overview and translational considerations. Neurosci Biobehav Rev. April; 87:233-254
Heckman P. R., van Duinen M. A., Bollen E. P., Nishi A., Wennogle L. P., Blokland A., Prickaerts J. Phosphodiesterase Inhibition and Regulation of Dopaminergic Frontal and Striatal Functioning: Clinical Implications. (2016) Int J Neuropsychopharmacol. April 30. pii: pyw030. doi: 10.1093/ijnp/pyw030
M. Hoenicka, E. M. Becker, H. Apeler, T. Sirichoke, H. Schroeder, R. Gerzer and J.-P. Stasch, “Purified soluble guanylyl cyclase expressed in a baculovirus/Sf9 system: Stimulation by YC-1, nitric oxide, and carbon oxide”, J Mol. Med. 77 (1999), 14-23
Prickaerts, J., Steinbusch, H. W. M., Smits, J. F. M., De Vente, J., (1997). Possible role of nitric oxide-cyclic GMP pathway in object recognition memory: Effects of 7-nitroindazole and zaprinast. Eur J Pharmacol., 337; 125-136
Reagan-Shaw, S., Nihal, M., Ahmad, N., (2007). Dose translation from animal to human studies revisited. FASEB J, 22; 659-661
Schmidt, H. H., Schmidt, P. M., and Stasch, J. P. (2009). NO- and haem-independent soluble guanylate cyclase activators. Handb Exp Pharmacol. (191):309-39
Stasch, J. P., Pacher, P., and Evgenov, O. V. (2009). Soluble guanylate cyclase as an emerging therapeutic target in cardiopulmonary disease. Circulation. 123(20):2263-73
Stasch, J. P., Hobbs, A. J. (2009). NO-independent, haem-dependent soluble guanylate cyclase stimulators. Handb Exp Pharmacol. 191:277-308
Vanmierlo, T., Rutten, K., Dederen, J., Bloks, V. W., van Vark-van der Zee, L. C., Kuipers, F., Kiliaan, A., Blokland, A., Sijbrands, E., Steinbusch, H., Prickaerts, J., Lutjohann, D., Mulder, M., (2011). Liver X receptor activation restores memory in aged AD mice without reducing amyloid. Neurobiol Aging., 32; 1262-1272.

1-14. (canceled)

15. A method of treating cognitive impairment in a mammal in need of such treatment, comprising administering to the mammal a therapeutically effective amount of a sGC activator or a pharmaceutically acceptable salt thereof, wherein the sGC activator is administered orally at a daily dose of 0.2 to 25 mg.

16. The method of claim 15, wherein the sGC activator is 3-(4-chloro-3-{[(2S,3R)-2-(4-chlorophenyl)-4,4,4-trifluoro-3-methylbutanoyl]amino}phenyl)-3-cyclopropylpropanoic acid of formula (1) 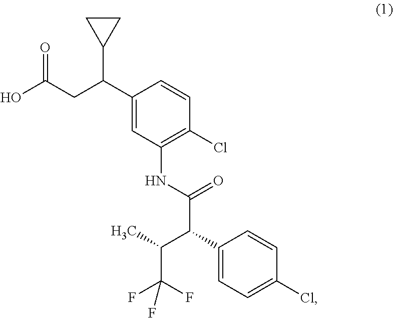 [SMILES: C[C@H]([C@H](C(=O)Nc1cc(C(CC(=O)O)C2CC2)ccc1Cl)c1ccc(Cl)cc1)C(F)(F)F]
or a pharmaceutically acceptable salt thereof.

17. The method of claim 15, wherein the orally administered daily dose is 1 to 10 mg.

18. The method of claim 15, wherein the orally administered daily dose does not significantly reduce blood pressure.

19. The method of claim 15, wherein cognitive impairment is selected from the group consisting of mild cognitive impairment, dementia, vascular dementia, Alzheimer dementia and cognitive impairment associated with cerebral infarctions, cerebral ischemia and ischemic stroke.

20. The method of claim 19, wherein cognitive impairment is vascular dementia.

21. The method of claim 16 further comprising administering to the mammal at least one compound selected from the group consisting of organic nitrates and NO-donors, inhibitors of phosphodiesterases 1, 2, 3, 4 or 5, anti-inflammatory compounds, immunosuppressive compounds, acetylcholinesterase inhibitors, NMDA receptor antagonists, compounds suitable for lowering blood pressure, antithrombotic compounds, compounds suitable for altering fat metabolism and antidiabetic compounds.

22. The method of claim 16 further comprising administering to the mammal at least one compound selected from the group consisting of donepezil, rivastigmine, galantamine and memantine.

23. A method of treating cognitive impairment in a mammal in need of such treatment, comprising administering to the mammal a therapeutically effective amount of methyl{4,6-diamino-2-[5-fluoro-1-(2-fluorobenzyl)-1H-pyrazolo[3,4-b]pyridin-3-yl]pyrimidin-5-yl}carbamate of formula (5) 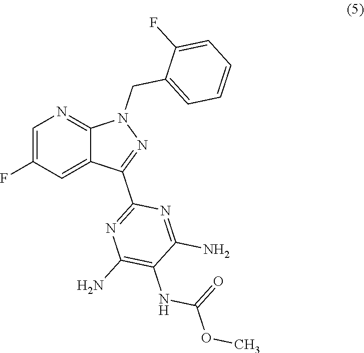 [SMILES: COC(=O)Nc1c(N)nc(-c2nn(Cc3ccccc3F)c3ncc(F)cc23)nc1N]
or a pharmaceutically acceptable salt thereof, wherein the compound of the formula (5) or a pharmaceutically acceptable salt thereof is administered orally at a daily dose of 1 to 6 mg.

24. The method of claim 23, wherein the compound of formula (5) or a pharmaceutically acceptable salt thereof is administered orally at a daily dose of 1 to 3 mg.

25. The method of claim 23, wherein the orally administered daily dose does not significantly reduce blood pressure.

26. The method of claim 23, wherein cognitive impairment is selected from the group consisting of mild cognitive impairment, dementia, vascular dementia, Alzheimer dementia and cognitive impairment associated with cerebral infarctions, cerebral ischemia and ischemic stroke.

27. The method of claim 26, wherein cognitive impairment is vascular dementia.

28. The method of claim 23 further comprising administering to the mammal at least one compound selected from the group consisting of organic nitrates and NO-donors, inhibitors of phosphodiesterases 1, 2, 3, 4 or 5, anti-inflammatory compounds, immunosuppressive compounds, acetylcholinesterase inhibitors, NMDA receptor antagonists, compounds suitable for lowering blood pressure, antithrombotic compounds, compounds suitable for altering fat metabolism and antidiabetic compounds.

29. The method of claim 23 further comprising administering to the mammal at least one compound selected from the group consisting of donepezil, rivastigmine, galantamine and memantine.

30. A method of treating cognitive impairment in a mammal in need of such treatment, comprising administering to the mammal a therapeutically effective amount of ent-N-[(2S)-amino-2-methylbutyl]-8-[(2,6-difluorobenzyl)oxy]-2,6-dimethylimidazo[1,2-a]pyridine-3-carboxamide (enantiomer A) of formula (6) 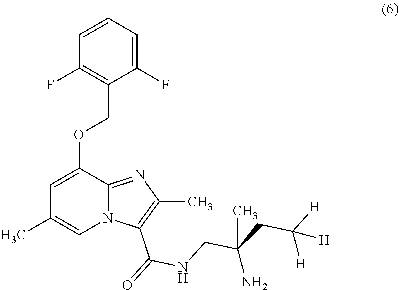 [SMILES: CC[C@@](C)(N)CNC(=O)c1c(C)nc2c(OCc3c(F)cccc3F)cc(C)cn12]
or a pharmaceutically acceptable salt thereof, wherein the compound of formula (6) is administered orally at a daily dose of 0.2 to 6 mg.

31. The method of claim 30, wherein the compound of formula (6) or a pharmaceutically acceptable salt thereof is administered orally at a daily dose of 0.3 to 3 mg.

32. The method of claim 30, wherein the orally administered daily dose does not significantly reduce blood pressure.

33. The method of claim 30, wherein cognitive impairment is selected from the group consisting of mild cognitive impairment, dementia, vascular dementia, Alzheimer dementia and cognitive impairment associated with cerebral infarctions, cerebral ischemia and ischemic stroke.

34. The method of claim 33, wherein cognitive impairment is vascular dementia.

35. The method of claim 30 further comprising administering to the mammal at least one compound selected from the group consisting of organic nitrates and NO-donors, inhibitors of phosphodiesterases 1, 2, 3, 4 or 5, anti-inflammatory compounds, immunosuppressive compounds, acetylcholinesterase inhibitors, NMDA receptor antagonists, compounds suitable for lowering blood pressure, antithrombotic compounds, compounds suitable for altering fat metabolism and antidiabetic compounds.

36. The method of claim 30 further comprising administering to the mammal at least one compound selected from the group consisting of donepezil, rivastigmine, galantamine and memantine.
